(19) United States
(12) Patent Application Publication
(10) Pub. No.: US 2019/0290215 A1
(43) Pub. Date: Sep. 26, 2019

(54) MEDICAL PROCEDURE LOGGING IN A COMPLEX MEDICAL PROCEDURE

(71) Applicant: Asia Pacific Medical Technology Development Company, Ltd, Central (HK)

(72) Inventor: John R. Gilbert, Brookline, MA (US)

(73) Assignees: Asia Pacific Medical Technology Development Company, Ltd, Central (HK); Asia Pacific Medical Technology Development Company, Ltd, Central (HK)

(21) Appl. No.: 16/086,249

(22) PCT Filed: May 11, 2016
(86) PCT No.: PCT/US2016/031727

§ 371 Date: Sep. 18, 2018

Related U.S. Application Data
(60) Provisional application No. 62/310,715, filed on Mar. 19, 2016.

(51) Int. Cl. A61B 5/00; A61B 1/04; G16H 10/60; G16H 30/40

(52) U.S. Cl. CPC A61B 5/743; A61B 1/04; A61B 5/7435; A61B 5/0402; G16H 10/60; G16H 30/40; A61B 5/746

(57) ABSTRACT

Methodologies and systems are provided for an operating location logging system for use in connection with a complex medical procedure. Example operating location logging systems include an image recording system configured to capture at least one image of a portion of at least one of a data recording object or a display of an instrument, and a console including at least one processing device. The at least one processing device is programmed to receive image data, representative of a plurality of images, that is transmitted using a data transmission component of the image recording system, and collect and/or compute medical data in connection with the complex medical procedure.

### RELATED APPLICATION

This application claims the benefit of and priority to U.S. Provisional Application No. 62/310,715, entitled “Dynamic Medical Procedure Logging,” filed Mar. 19, 2016, which is hereby incorporated by reference in its entirety.

### BACKGROUND

The many different instruments used in an operating room provide data pertinent to a medical procedure being performed on a patient. The medical personnel in the operating room have knowledge about the type of data they need from the instruments and at what point in time during the procedure they need that data. At least one surgical assistant in the operating room can be tasked to walk around the operating room, review the instruments, and write notations to clipboards or logbook to provide additional information in connection with the medical procedure. To improve on the performance of the medical procedure, the surgeon or other medical personnel in the operating room may need data with greater frequency than can be collected by the medical assistant, or may need analysis of the data at timescales that the surgical assistant would not be able to provide.

### SUMMARY

In accordance with embodiments of the present disclosure, operating location logging systems and control systems are described.

Example systems, methods, and apparatus provide an operating location logging system for use in a complex medical procedure. An example system can include an image recording system configured to capture an image of at least a portion of at least one of a display of an instrument or a data recording object, a console, a database and at least one processing device. The example image recording system includes an image capture device, a mounting device coupled to the image capture device, and a data transmission component. The example mounting device is configured to mount the image recording system relative to the data recording object or the display of the instrument. In the example systems, methods, and apparatus, the at least one processing device is programmed to cause the data transmission component to transmit image data representative of at least one image of a plurality of images captured using the image capture device, associate a timestamp with the at least one image of the plurality of images, each timestamp being representative of a time of capture of the at least one image, and associate operator level data with at least one region of interest of the at least one image, the operator level data comprising at least one of: a procedure event data, a procedure criterion, a procedure step, a region of interest identifier, or an instrument identifier. The at least one processing device is also programmed to store the image data associated with the respective at least one region of interest of the at least one image and the associated respective timestamp in the database as patient medical data. The example console is configured to display the at least one image of the plurality of images on a display unit and render a graphical user interface. The example graphical user interface can includes at least one of a first interactive user interface element configured for receiving a user indication of previously stored at least one region of interest and associated operator level data to be retrieved, or a second interactive user interface element configured for receiving a user indication of previously stored patient medical data to be retrieved, or a third interactive user interface element configured for receiving a user indication of the at least one region of interest of the at least one image of the plurality of images and a fourth interactive user interface element for receiving the operator level data corresponding to the at least one region of interest of the at least one image.

Example systems, methods, and apparatus provide an operating location logging system for use in a complex medical procedure. An example system can include an image recording system configured to capture an image of at least a portion of at least one of a display of an instrument or a data recording object, a console, a database and at least one processing device. The example image recording system includes an image capture device, a mounting device coupled to the image capture device, and a data transmission component. The example mounting device is configured to mount the image recording system relative to the data recording object or the display of the instrument. In the example systems, methods, and apparatus, the at least one processing device is programmed to cause the data transmission component to transmit image data representative of at least one image of a plurality of images captured using the image capture device, associate a timestamp with the at least one image of the plurality of images, each timestamp being representative of a time of capture of the at least one image, and associate operator level data with at least one region of interest of the at least one image, the operator level data comprising at least one of: a procedure event data, a procedure criterion, a procedure step, a region of interest identifier, or an instrument identifier. The at least one processing device is also programmed to extract procedure instrument data and/or procedure event data from the image data corresponding to the at least one region of interest, the procedure instrument data and/or the procedure event data including one or more of: alphanumeric data, data indicative of a graphical feature associated with an image object in the region of interest, or data indicative of a notation made in the region of interest, and store the at least of the extracted procedure instrument data, or the extracted procedure event data, or the at least one medical procedure parameter, and the associated respective timestamp in the database as patient medical data. The example console is configured to display the at least one image of the plurality of images on a display unit and render a graphical user interface. The example graphical user interface can includes at least one of a first interactive user interface element configured for receiving a user indication of previously stored at least one region of interest and associated operator level data to be retrieved, or a second interactive user interface element configured for receiving a user indication of previously stored patient medical data to be retrieved, or a third interactive user interface element configured for receiving a user indication of the at least one region of interest of the at least one image of the plurality of images and a fourth interactive user interface element for receiving the operator level data corresponding to the at least one region of interest of the at least one image. The example console is also configured to display a rendering of the at least one graphical representation of the at least one medical procedure parameter.

Example systems, methods, and apparatus provide an operating location logging system for use in a complex medical procedure. An example system can include an image recording system configured to capture an image of at least a portion of at least one of a display of an instrument or a data recording object, a console, a database and at least one processing device. The example image recording system includes an image capture device, a mounting device coupled to the image capture device, and a data transmission component. The example mounting device is configured to mount the image recording system relative to the data recording object or the display of the instrument. In the example systems, methods, and apparatus, the at least one processing device is programmed to cause the data transmission component to transmit image data representative of at least one image of a plurality of images captured using the image capture device, associate a timestamp with the at least one image of the plurality of images, each timestamp being representative of a time of capture of the at least one image, and associate operator level data with at least one region of interest of the at least one image, the operator level data comprising at least one of: a procedure event data, a procedure criterion, a procedure step, a region of interest identifier, or an instrument identifier. The at least one processing device is also programmed to extract procedure instrument data and/or procedure event data from the image data corresponding to the at least one region of interest, the procedure instrument data and/or the procedure event data including one or more of: alphanumeric data, data indicative of a graphical feature associated with an image object in the region of interest, or data indicative of a notation made in the region of interest, compute at least one medical procedure parameter using the procedure instrument data and/or the operator level data, and the associated timestamp, generate at least one graphical representation of the at least one medical procedure parameter, and store the at least of the extracted procedure instrument data, or the extracted procedure event data, or the at least one medical procedure parameter, and the associated respective timestamp in the database as patient medical data. The example console is configured to display the at least one image of the plurality of images on a display unit and render a graphical user interface. The example graphical user interface can includes at least one of a first interactive user interface element configured for receiving a user indication of previously stored at least one region of interest and associated operator level data to be retrieved, or a second interactive user interface element configured for receiving a user indication of previously stored patient medical data to be retrieved, or a third interactive user interface element configured for receiving a user indication of the at least one region of interest of the at least one image of the plurality of images and a fourth interactive user interface element for receiving the operator level data corresponding to the at least one region of interest of the at least one image. The example console is also configured to display a rendering of the at least one graphical representation of the at least one medical procedure parameter.

Example systems, methods, and apparatus provide a control system for use in a complex medical procedure. An example system can include an image recording system configured to capture an image of at least a portion of a display of a sense medical instrument that is in communication with at least a first portion of a body, a drive medical instrument in communication with at least a second portion of the body, and at least one processing device. The example image recording system includes an image capture device, a mounting device coupled to the image capture device, and a data transmission component. The example mounting device is configured to mount the image recording system relative to the display of the sense medical instrument. In the example systems, methods, and apparatus, the at least one processing device is programmed to cause the data transmission component to transmit image data representative of at least one image of a plurality of images captured using the image capture device, extract procedure instrument data from the image data corresponding to a region of interest of at least one image of the plurality of images, the procedure instrument data comprising data representative of at least one measurement of the sense medical instrument, compute a control signal based at least in part on the extracted procedure instrument data to cause the drive medical instrument to maintain or change an operation setting, and transmit the control signal to the drive medical instrument.

Additional combinations or permutations of the above examples are envisioned as being within the scope of the present disclosure. It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

### BRIEF DESCRIPTION OF THE DRAWINGS

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally similar and/or structurally similar elements).

The foregoing and other features and advantages provided by the present disclosure will be more fully understood from the following description of exemplary embodiments when read together with the accompanying drawings, in which:

FIGS. 1A-1E show non-limiting examples of medical instruments and instrument displays that can be used in a complex medical procedure, according to principles of the present disclosure.

FIGS. 1F-1G show examples of clipboards for use in connection with a complex medical procedure, according to principles of the present disclosure.

FIG. 1H shows an example of a logbook for use in connection with a complex medical procedure, according to principles of the present disclosure.

FIG. 2A shows an example of operating locations associated with the stages of a complex medical procedure, according to principles of the present disclosure.

FIG. 2B shows an example of an operating location logging system in an operating room or other medical facility, according to principles of the present disclosure.

FIG. 2C shows an example console and example medical instruments mounted to a mobile cart, according to principles of the present disclosure.

FIG. 3 shows a block diagram of an example operating location logging system, according to principles of the present disclosure.

FIGS. 4A-4D show example image recording systems mounted relative to objects or medical instruments, according to principles of the present disclosure.

FIGS. 5A-5D show screens of an example console of an operating location logging system, according to principles of the present disclosure.

FIGS. 6A-6B show screens of another example console of an operating location logging system, according to principles of the present disclosure.

FIG. 7 shows a screen of another example console of an operating location logging system, according to principles of the present disclosure.

FIG. 8A-8B show screens of an example console of an operating location logging system, according to principles of the present disclosure.

FIG. 8C shows an example plot of temperature measurements versus time since cardiac arrest for a patient, according to principles of the present disclosure.

FIGS. 9A-9B show example control systems configured for closed loop operation, according to principles of the present disclosure.

FIG. 10 shows a block diagram of an example computing device that can be used as a console to perform example processes, according to principles of the present disclosure.

FIG. 11 shows a diagram of an example network environment suitable for a distributed implementation, according to principles of the present disclosure.

### DETAILED DESCRIPTION

Following below are more detailed descriptions of various concepts related to, and embodiments of, inventive methods, apparatus, and systems for logging activities, procedures, and results of a complex medical procedure. It should be appreciated that various concepts introduced above and discussed in greater detail below may be implemented in any of numerous ways, as the disclosed concepts are not limited to any particular manner of implementation. Examples of specific implementations and applications are provided primarily for illustrative purposes.

As used herein, the term “includes” means includes but is not limited to, the term “including” means including but not limited to. The term “based on” means based at least in part on.

Example methodologies, systems, apparatus, and non-transitory computer-readable media described herein provide an operating location logging system that can be used in connection with a medical procedure. The operating location logging system includes a plurality of image recording systems that are configured to mount to any number of differing types of objects or instruments used in the medical procedure, and a console that coordinates with the image recording systems. The console of the example operating location logging system can be used to log activities and identify and record medical events occurring during the medical procedure, based on an analysis of timestamped image data from regions of interest of the images captured using the image recording system and operator level data received in connection with the medical procedure. The example operating location logging system does not rely on any given instrument or object being configured or configurable to communicate with the console. As a result, the operating location logging system can be implemented widely in may differing types of medical situations, to perform any of the processes and methodologies described herein, regardless of the type of manufacturer or format of any given medical instrument or data recording object, i.e., whether a paper object, or an analog or digital instrument.

Example medical instruments according to the principles herein can be configured with sensing functions (sense medical instruments) and/or with driving functions (drive medical instruments). Example medical instruments with sensing functions can be probes, or coupled to probes, in communication with at least a portion of the patient body. Non-limiting examples of sense medical instruments include instruments that are configured to measure temperature, pressure, liquid volume flow rate, blood oxygen concentration, pH, vibration, heart rate, and voltage signals in electrocardiography or electroencephalography. Example medical instruments with driving functions can be disposed in communication with at least an application system coupled to at least portion of the patient body, for applying a stimulus, an energy or a force, or for administering a material or chemical, to at least portion of the body. As a non-limiting example, the drive medical instrument can be a blood pump that drives an extracorporeal blood circuit pumping blood in and out of the body. As a non-limiting example, the drive medical instrument can be a water pump that pumps cold water through a heat exchanger to cause blood flowing through that extracorporeal circuit to be cooled, a water pump that pumps warm water through a water-blanket causing a patient to the heated by thermal conduction through their skin, a syringe pump driving a controlled flow rate volume of saline solution including a drug into an intravenous catheter and into the patient, an electrical stimulation device used to apply a voltage to an electrode at the patient wrist to stimulate the median sensory nerves in order to observe a somatosensory response during intra-operative monitoring.

In various non-limiting examples, the drive medical instrument can be in communication with at least a portion of the body via an application device. As a non-limiting example, where the drive medical instrument is used to apply a stimulus (including electrical or temperature), an energy or a force (including pressure) to a portion of the body, the application device may be, for example, a set of electrodes mounted to the body, a lever in contact with the body, a needle, a thermal wrap, a pressure cuff, or other device. In another non-limiting example, where the drive medical instrument is used to administer a material or chemical to the body, the application device may be a needle, a syringe, a hose, or other device. In any example herein, the application device is in a form configured to communicate the desired effect of the drive medical instrument to the body. As another example, where the drive medical instrument is a water heater/chiller, the application device can be a water blanket mounted on the patient. As yet another example, where the drive medical instrument is a blood pump, the application device can be a needle. As yet another example, where the drive medical instrument is an electrical stimulation device, the application device can be electrodes.

An instrument display for a medical instrument with sensing functions can show values of the sensors. An instrument display for a medical instrument with driving functions can have regions that show the value of the set points of the drive medical instrument, or value of the stimulus, force, or energy being applied, or values of the amount of a material or chemical being administered to the patient using an application system.

According to the principles herein, a sense medical instrument has at least one sensing function, and a drive medical instrument has at least one driving function.

In some examples, the medical instruments can have both sensing and driving functions. In a non-limiting example, instrument displays for pump systems can indicate or display sensed values of flow rate or pressure observed, as well as values of set point of flow rate applied to the fluid circuit to which they are coupled. Example medical instruments herein may not specifically attach to a portion of the patient body, but can be used to monitor or control the operating room or some other aspect of the operation that may be of interest to the operators. For example, elapsed time or operating room power stability, or air conditioning quality may also be monitored and logged using medical instruments.

In non-limiting examples, the operating location has many different medical instruments that provide medical data pertinent to the complex medical procedure. Many of the medical instruments are derived from several different instrument manufacturers. Some medical instruments are old and some instruments are newer. Some medical instruments are configured, or could be configured or retro-fitted, to transmit data. Some medical instruments cannot be configured to transmit data.

Example methods, apparatus, and systems described herein provide for logging activities, procedures, and results of a complex medical procedure performed on a patient. According to the principles herein, a complex medical procedure is a course of action performed by a plurality of medical operators on a patient, where the medical procedure involves a plurality of stages using a plurality of different medical instruments that provide data pertinent to the medical procedure to the medical operators.

In a complex medical procedure, at least one of the medical operators performs a logging or recording of at least one medically-significant event that takes place in connection with the complex medical procedure, where at least one of the medical operators classifies an event as a medically-significant event based on trained medical judgment of the medical significance.

In different examples herein, the patient can be a human or a non-human animal.

In the example systems, methods, and apparatus according to the principles herein, a medically-significant event can be an occurrence prior to, during, or after a complex medical procedure that is sufficiently clinically relevant to be desirable to record. Non-limiting examples of medically-significant events include, but are not limited to, descriptions of the actions of the medical operators, and/or the data and other readings of the medical instruments, and/or observations about the status or other condition of the patient made by at least one of the medical operators.

Stages of the example complex medical procedure are performed by one or more medical operators. Example medical operators can be in a team together performs a stage of the complex medical procedure, or the entire complex medical procedure. Such a team can include medical operators who are not all required to have the same training, and each can be appropriately trained in at least their own tasks in the procedure. At least one member of the team can be in control at any stage of a procedure. Typically the lead medical operator is a doctor or surgeon having the appropriate level of training. The medical operators in the operating location have knowledge about the type of data they need from each instrument or object, and at what point in time during the procedure they need that data. This includes the data from writing or notations made to objects such as clipboards and logbook in connection with the medical procedure.

At least one team member can have the responsibility of logging or recording the medically-significant events (as described hereinabove) of the procedure at any given stage or phase of the procedure. For example, a surgical assistant may be tasked with walking around the operating location at regular time intervals (e.g., every 30 minutes) and making notes on data from each instrument display, or concerning certain events occurring during the medical procedure. However, the surgeon or other practitioner in the room may need data with greater frequency than these regular intervals, or may need analysis of the data that the surgical assistant could not provide. Furthermore, in the amount of time it takes for the surgical assistant to complete the circuit around the room, data may be gone from an instrument display which could be valuable for making decisions concerning the medical procedure. The example systems, methods, and apparatus can provide for automation of the logging of data from an instrument display, which can be of great value and can improve efficiency and the results of the complex medical procedure.

Example medical instruments can be used for different purposes, and can come from many different manufacturers. Medical instruments used in a complex medical procedure may be a mix of older or outdated instruments and newer instruments. Some example medical instruments may have no capabilities for wired or wireless output of data. Other example medical instruments may have capabilities for output of wired or wireless data, but it would not be available in real-time. Yet other example medical instruments may have capabilities to output data, but may use such incompatible formats or data models that expensive special consultants or technicians (usually not found among the medical operators) would be required to log data from these medical instruments or to integrate them with other medical instruments.

There previously has been no workable and universally available standards for information interchange that allow real time data from these instruments to be collected and logged in operating locations. That is, in practice, operating locations today have one or more instruments that are either incapable of communicating in real time, or for which it is too difficult or expensive to integrate them. The example systems, methods, and apparatus described herein provide means collecting data from this wide variety of instruments and instrument types.

In particular, the example systems, methods, and apparatus described herein can be applied over a wide variety of instruments found in operating locations that have a user display.

The example instruments can share the characteristic that they display some data to the medical operators, where the data can be pertinent to the performed medical procedure or is otherwise of interest to the medical operator(s). Non-limiting examples of data types that might be displayed by medical instruments include, but are not limited to, data about the instrument status, sensor data about the patient, or data about the instruments performance of some actuation or effect on the patient. An example of the instrument status can be, but is not limited to, an indication that the instrument is functioning properly or that the power is on. An example of the sensor data about the patient can be, but is not limited to, an indication a temperature at a left nasal probe, or pressure at the tip of a catheter. An example of the instruments performance of some actuation or effect on the patient can be, but is not limited to, an indication of the flow rate set point of blood pump in an extracorporeal circuit or a set point of temperature of a water heater/chiller driving a water blanket on the patient.

In non-limiting examples, the medical instruments can include embedded controllers (including central processing units) that may have many internal software variables. Instrument designers make decisions, based on their knowledge of the intended instrument user, about what data to display on the display of the instrument and what data the instrument may have internally (software variables in the case of embedded controllers) that is not displayed. In various examples, data in connection with the medical operator in the specified use of the instrument can be displayed on the display, and data that may not be relevant to the medical operator in the specified use of the instrument may not be displayed (for example, to avoid user confusion). In the case of instruments in connection with medical procedures on human patients, the instrument display can be design and configured to display data based on regulations, such as but not limited to those from the U.S. Food and Drug Administration or the Medical Directives of the Conformité Européenne (CE) system.

Another aspect of current medical instrument design is that few instruments are designed with one very specific procedure in mind. Instead, they are designed to be used in as many different procedures as possible. In a non-limiting example, a pressure measurement instrument can accept a pressure sensor probe input and display for the user the pressure in mmHg units corresponding to the pressure at the tip of the probe. If the pressure sensor probe is located at the tip of a percutaneous interventional catheter, such a system might be used in interventions for coronary angioplasty or coronary stenting or angioplasty interventions in the leg for peripheral arterial disease (PAD) or localized drug delivery operations for applying chemotherapeutic compounds to tumor bearing organs. In each case the site of the pressure sensor probe for performing the pressure measurement is defined by the location into which the pressure sensor probe is placed in the body, such as but not limited to, the heart, leg, liver, or kidney.

The example systems, methods, and apparatus provide means for collecting medical data in connection with the complex medical procedure. Medical data can be procedure instrument data and/or operator level data. As described in greater detail hereinbelow, procedure instrument data can include data presented on the displays of the instruments used in the complex medical procedure. As also described in greater detail hereinbelow, the operator level data includes data that is useful to a medical operator but that is not present on an instrument display, including procedure event data, data indicating the region of interest of an image of an instrument display that would be of interest to a medical operator, and data indicating the procedure criteria for the medical procedure.

As used herein, “procedure instrument data” refers to data presented on the display of an instrument used in the procedure. Non-limiting examples of procedure instrument data include data from instrument displays of a blood pump, a temperature monitor attached to a temperature probe, an electrocardiogram (ECG) system, a drug infusion system, a fluoroscopy system, a heart monitor, a hypothermia system, or other instrument. The procedure instrument data that can be derived from the display of these instruments include numbers, text, images, graphs, status icons, or other data. In various examples, the instrument display may be co-located with the bulk of the instrument, or may be located remote from the instrument. As a non-limiting example, a chiller might have a physical box including a thermoelectric cooler and a water pump, and transmit the flow rate of the pump wirelessly to a dedicated screen (i.e., instrument display) for display of that flow rate, or that display might be mounted on the surface of the physical box, or that display module might be coupled to the physical box only by a data transmission cable.

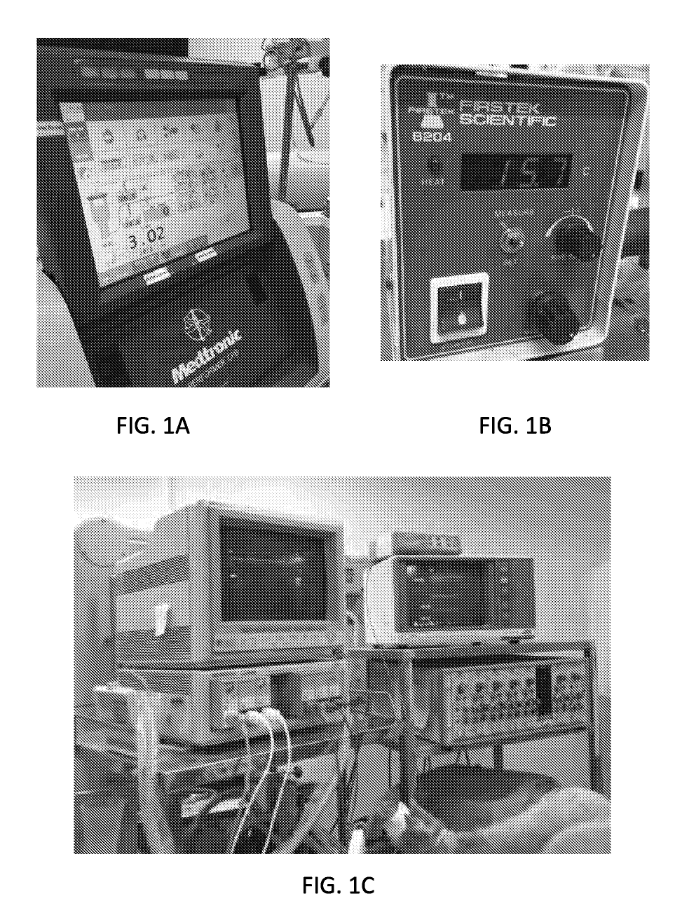

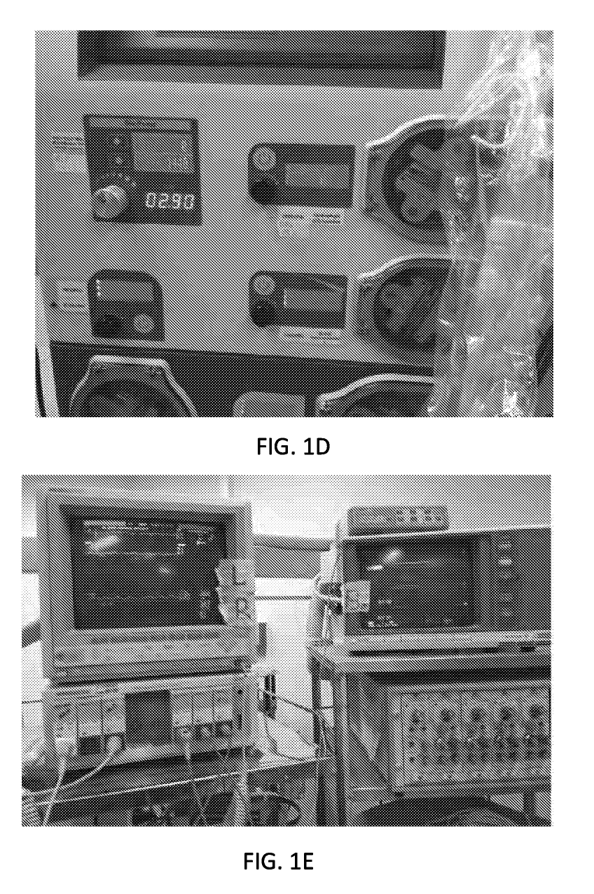

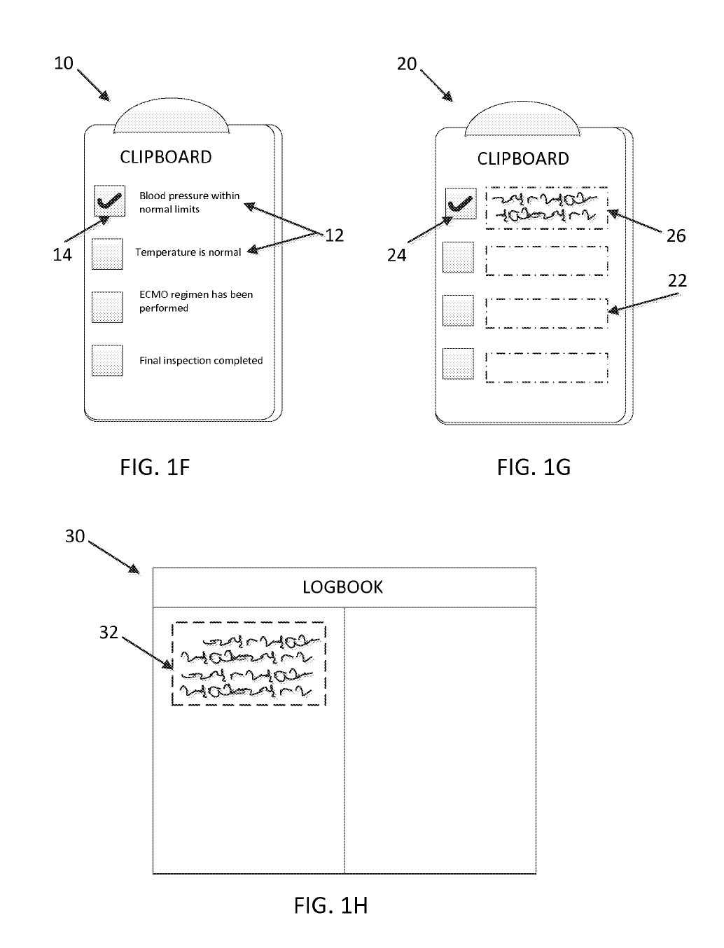

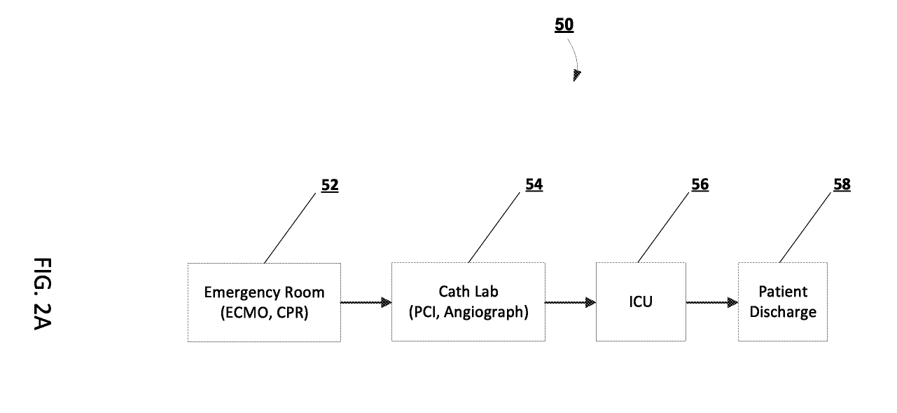

FIGS. 1A-1E show non-limiting examples of instrument displays of medical instruments that can be used in a complex medical procedure. FIG. 1A shows an instrument display of a Medtronic Performer™ cardiopulmonary bypass (CPB) system available from Medtronic, Inc. FIG. 1B shows a FIRSTTEK™ heater-chiller by Firstek Scientific (Taipei, Taiwan). FIG. 1C shows example of temperature and pressure interfaces. FIG. 1D shows an instrument display for rotary pumps. FIG. 1E shows example instrument displays for temperature and blood pressure measurements.

In an example, instrument display may display data directly to a user, or may include relay(s) to relay data from the medical instrument to the user. For example, an image from an x-ray film mounted to a light box (or other illuminating device) may be relayed to a monitor, such that the monitor serves as the instrument display.

As used herein, “operator level data” refers to data that is useful to a medical operator but that is not present on an instrument display. Operator level data encompasses data that is sufficiently significant to log or monitor in the trained medical judgment of a medical operator.

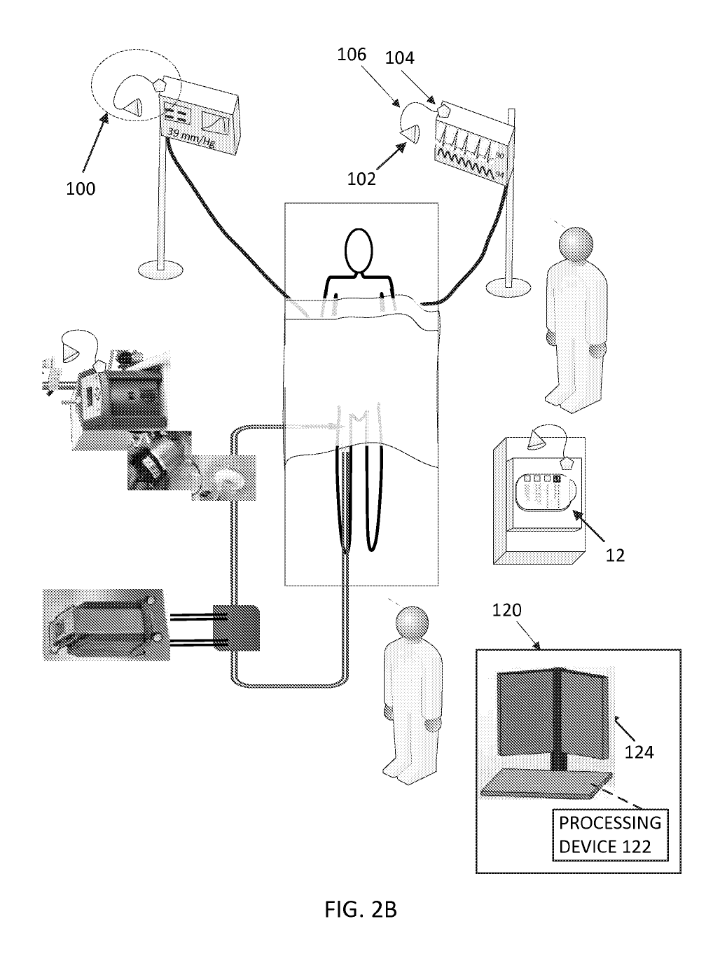

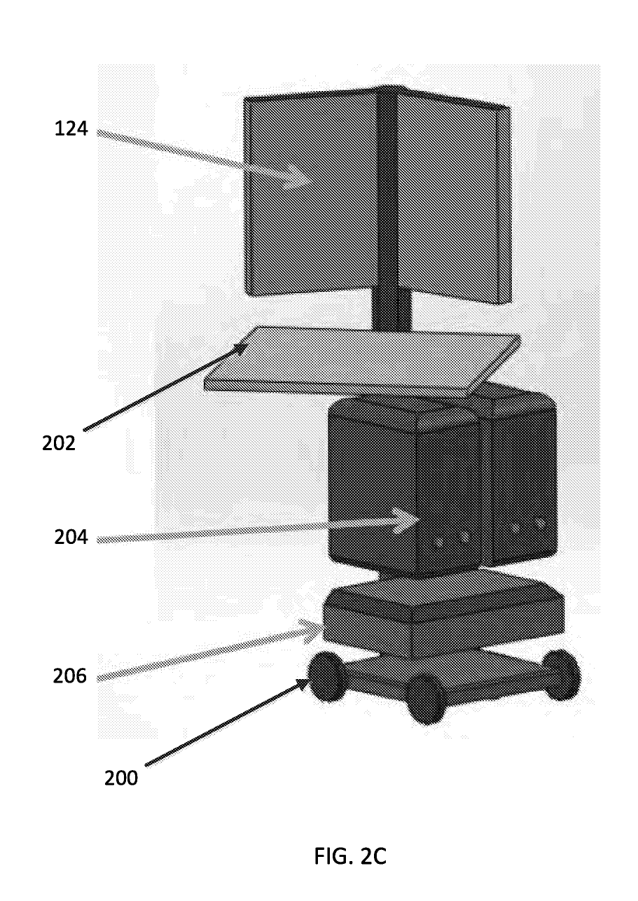

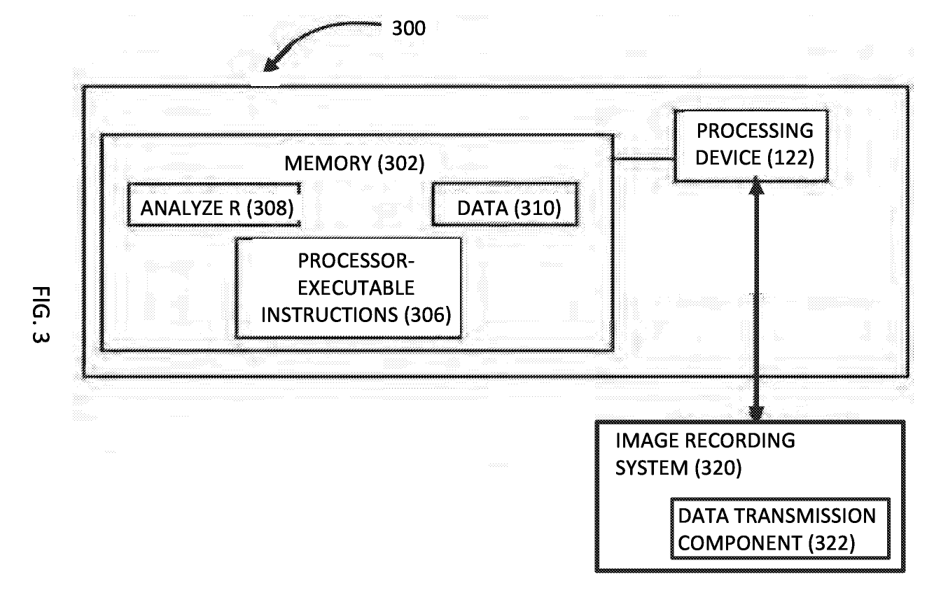

Non-limiting examples of operator level data include the regions of interest of images taken of at least a portion of a display of a medical instrument or a data recording object, identifiers of the regions of interest, identifiers of the medical instruments, the procedure event data, data representative of procedure steps, and the procedure criteria. The operator level data includes information the medical operator of a medical instrument records about the medical instrument. For example, a medical operator may indicate, using a selection tool shown as a rectangle or other polygonal shape on an image of the display of the medical instrument, a region of interest that encompasses a number, which can be, but is not limited to, a value of a pressure reading of a pressure monitor. The medical operator may also associate an identifier with the region of interest, such that the identifier indicates what the region of interest means to the medical operator. For example, the region of interest of an image of the instrument display of a pressure monitor may be associated with an identifier as the “left wrist pressure in mmHg”. In some examples, the region of interest and its associated identifier may be entered by the medical operator at a console of an example system (described in greater detail hereinbelow in connection with FIGS. 2B-11). The region of interest and its associated identifier may be indicated at the console at the beginning of a complex medical procedure, or may be recalled from a computer-readable medium in a setup phase prior to the commencement of a complex medical procedure.

In another non-limiting example, the operator level data can be logging instructions that specify when image data is to be transmitted from the image recording system to the console, for association with timestamp and other operator level data as described herein. There can be differing logging instructions for each different image capture device of the image recording system. The logging instructions associated with a given image capture device for use during a given stage, or multiple stages, of a complex medical procedure may be used in more than one procedure program, and therefore may be stored (e.g., in a database) and recalled. In some examples, the data transmission components associated with the various image capture devices can be caused to transmit image data at regular time intervals, such as but not limited to every minute, or every 30 seconds, or other appropriate fixed rate, as specified by an operator in the logging instructions. In some examples, the data transmission components associated with the various image capture devices may be caused to transmit the image data at different constant rates based on the logging instructions. In some examples, the logging instructions can specify that the image capture system update the captured images based on a determination of a change of the image being captured, such as but not limited to a change of the instrument display or object being monitored.

In another non-limiting example, the operator level data can be an instrument identifier, such as data indicating the role the medical instrument in at least one step of the complex medical procedure, or in a particular step of procedure, or data indicating where a medical instrument is disposed, such as but not limited to a location of placement on a patient of each of a plurality of sensors. For example, the instrument identifier or the region of interest identifier can be used to differentiate between a temperature sensor probe that is coupled to a left nasal location and a temperature sensor probe that is coupled to the bladder. As yet another example, the instrument identifier or region of interest identifier can be used to indicate what a medical operator wants to know from each region of interest indicated on each images, and the data types of the associated procedure instrument data or procedure event data. The operator level data pertaining to the location of positioning of a medical instrument, such as but not limited to a pressure sensor probe, may not be indicated as part of the instrument data shown on the instrument display, or even be noted within any embedded controller (if present). Rather, that data may be provided by at least one medical operator of the medical instrument (such as the medical operator who disposed the pressure sensor probe on the patient). Additional supplemental data about the medical instrument may also be added in this operator level data type, for example, a notation may be made to indicate whether the task of disposing the pressure probe on the patient was unusually easy or difficult.

In another non-limiting example, the operator level data includes data representative of procedure steps. The procedure steps include the expected sequence of events of a complex medical procedure. In this example, the operator level data can include data about alternative sequence branches in the sense of if-then- else patterns. For example, if a medical operator adds a vasodilation promoting drug to increase the blood flow to a patient periphery, then the region of interest of an image of the instrument display of the syringe pump supplying the vasodilator can be recorded. As another example, if the region of interest of an image of the instrument display of the temperature of the left nasal probe on a patient indicates that the temperature is returned to normothermia, then this can be an indicator that re-warming phase of the medical procedure is ended. A region of interest of an image of an instrument display for an instrument that is designated as relevant to the rewarming phase can be no longer monitored or may be shut off. In an example with medical instruments that are actuators (including instruments that move or control a mechanism or system), the operator level data representative of the procedure steps can include settings or instructions for the operation of the actuator. In a non-limiting example, a procedure step can be a rewarming step in a complex medical procedure involving hypothermia. In this example, the procedure step may specify an increase in the water heater/chiller temperature by about 0.5° C. per hour until normothermia is restored based on an analysis of data indicative of the bladder temperature. To effect this example procedure step, instruction may be sent directly from the console to a controller for a water heater/chiller controller or may be displayed on the console or otherwise communicated to a medical operator with a recommendation for the medical operator to input the instruction for this procedure step manually into the controller for the heater/chiller.

As used herein, “procedure event data” refers to data about events that happen during the medical procedure. As pointed out hereinabove, the procedure event data is a type of operator level data. Medical operators record their observations or actions taken (events) during the complex medical procedure using standard methods (including clipboards, logbooks, tablets, or other electronic data recording device). The example systems herein provide for improved logging of such events. For example, the procedure event data can be derived from check-marks, writings, or other notation made to objects used in the medical procedure for recording data, such as but not limited to clipboards and logbooks. Non-limiting examples of procedure event data include data indicating that a patient has reached an operating location, whether a patient is moved to an intensive care unit, a moment that a catheter is inserted, whether the patient's skin is sealed over an incision, whether a patient experienced a downturn in condition, etc. The timestamp associated with the image of a clipboard, or logbook, or other recording material also can be used to define the sequence of events represented in the procedure event data.

The example procedure event data represents the medically-significant data observed about the patient. The example procedure step indicates the medically-significant steps that a medical operator expects to perform on the patient in an example complex medical procedure. In a non-limiting example, both the procedure event data and the data representative of the procedure step may be verified based on an analysis of a recording of at least one image of a clipboard and a timestamp associated with the actual event. However, they may not be the same. For example, the data representative of a procedure step may be reusable with two or more different patients, while the procedure event data based on observations of what happens to a first patient patient can be different than that for a second patient.

FIGS. 1F-1H show non-limiting examples of objects (clipboards 10 and 20 and logbook 30) for use in connection with a complex medical procedure, according to the principles of the disclosure. As shown in FIG. 1F, an example clipboard 10 can include a list 12 of specified activities 12 that are to be performed in connection with the complex medical procedure, and that provide the procedure event data. The clipboard 10 also includes check fields 14 where notations (such as but not limited to check-marks) can be made by at least one medical operator, to indicate commencement or completion of each of the activities in the list 12. FIG. 1G shows another example clipboard 20 that can include a set of activity fields 22 in which at least one medical operator can make notations to indicate the activities being performed in connection with the complex medical procedure, to provide the procedure event data. The clipboard 20 also includes check fields 24 where notations (such as but not limited to check-marks) can be made by at least one medical operator, to indicate commencement or completion of the activities indicated in the corresponding activity field 22. FIG. 1G also shows an example of a completed activity field 26, showing hand-written notations made by a medical operator to indicate a medically-significant event. FIG. 1H shows an example logbook 30 that can be used by at least one medical operator to make notations concerning a medically-significant event in connection with the complex medical procedure, such as but not limited to a block 32 of hand-written text.

An example system according to the principles herein can be configured to record a timestamp to associate with the procedure event data of clipboard 10 or 20 or logbook 30, based on either a time that the notation is made to check field 14 or 24, a time that the hand-written notation is made to generate the completed activity field 26, or a time associated with the block 32 of hand-written text. For example, the entire block 32 of hand-written text can be timestamped based on the time that the first word of the paragraph is noted to the logbook (rather than multiple timestamps being associated if the hand-written notations take a certain amount of time). This allows text block 32 of handwriting to be associated with a single timestamped, and as a result the entire text block 32 can be recalled from a system log based on the timestamp associated with the medically-significant event that caused the medical operator to start making the notations of the text block that records the event.

As an example of timestamping, the procedure event data could define a time T1 that a catheter is inserted, a time T2 that sedation is increased, a time T3 that a temperature of injected blood is increased in a planned, controlled re-warming phase. As another example of timestamping, the procedure event data could provide a record of a timestamp T1 at which a checkmark is made to a clipboard to indicate that a catheter is inserted, a timestamp T2 at which a checkmark is made to the clipboard to indicate that sedation is increased, or a timestamp T3 at which a checkmark to a clipboard to indicate that the temperature of injected blood is increased at start of a planned, controlled re-warming phase.

As used herein, “procedure criteria” are a type of operator level data that indicates threshold conditions or expectations of the medical operators for the collected data and the course of events that may (or may not) occur during the complex medical procedure. In some examples, data indicating the procedure criteria can be entered by a medical operator at a user interface of an example console of an example system according to the principles herein. The example system also can be configured to apply the procedure criteria to evaluate the medical data collected or computed in connection with the complex medical procedure, and to generate alerts (such as notifications or alarms) to the medical operators to indicate whether the medical data from the complex medical procedure satisfies (or does not satisfy) the procedure criteria. A non-limiting example of use of a procedure criterion for a region of interest of an image could be to cause the example system to issue a notification if the temperature data shown in the region of interest of an image of an instrument display of a left nasal probe falls below about 20° C.. Another non-limiting example of use of a procedure criterion for a procedure event data could be to cause the example system to issue a notification if no notation had been made to the check field on a clipboard for a medically-significant event within a certain period of time (such as but not limited to no notation to indicate initiation of a ‘start rewarming’ action by about the 12-hour mark). In some examples, the procedure criteria may be based on “if-then-else” logic conditions, or based on evaluating procedure instrument data or procedure event data.

In an example, medical data pertaining to the location of positioning of a medical instrument, such as but not limited to a pressure sensor probe, may not be indicated as part of the instrument data shown on the instrument display, or even be noted within any embedded controller (if present). Rather, this type of operator level data is known to the medical operators of the medical instrument. This sub-type of operator level data can be classified as observation identity or naming data.

Example systems, methods, and apparatus described herein also allow logging of data available to the medical operators, but not the instruments, to be associated and recorded with the medical data collected from the instrument display.

In a non-limiting example of a sensor-type instrument, the medical operator in an oncology procedure may place the catheter tip at an artery feeding blood to the liver, and the medical data associated with the pressure measurement data is an indication that it is liver access pressure. In another non-limiting example of a sensor-type instrument, the medical operator may place the catheter tip at an artery feeding blood to the kidney, and the medical data associated with the pressure measurement data is an indication that it is kidney access pressure.

In some example systems, methodologies and apparatus according to the principles described herein, the event recording and logging function for instrument readings in an instrument display for a given team member or members may be eased so that event logging can be faster, more reliable, easier to perform and more configurable and customizable by the medical operators.

A non-limiting example of an actuator type instrument is a simple syringe drive for injecting a drug into the patient. The example syringe drive can have a setting and display showing its flow rate in units of ml/min. In this example, the medical data associated with the instrument data indicating the flow rate at each measurement during the procedure can include an indication of the location of the attachment point to the patient (for example an intra-venous line into the arm or leg or an intra-arterial line into the carotid artery), and the name of the drug and its concentration in units/ml in the syringe.

In a non-limiting example, the medically-significant events occurring in connection with the complex medical procedure, which are not measurable or recordable by medical instruments, are recorded by means of paper notebooks, logbooks, or clipboards. Examples of such events include data and notation characterizing certain actions taken by the medical operators in connection with the medical procedure, and/or data characterizing observations made by the medical operator(s) about the status or other condition of the patient. In some more modern facilities, these data recording objects might also be tablets (for handwriting recording) or keyboards (for text entry). The entries and notations by the medical operator can be used to provide data indicating the layer of medical judgment about the level of significance of the events occurring to the complex medical procedure.

In example implementations, the medical operators are considered to possess trained knowledge about the type of data need from each instrument or object, and at what point in time during the complex medical procedure that data is needed. This includes the data in connection with events, as may be recorded by writing or notations made to objects such as clipboards, notebooks, and logbooks in connection with the medical procedure.

The example systems, methods, and apparatus described herein provide means for collecting medical data from the medical instruments regardless of the physical location of the medical instrument or parts of the medical instrument.

A complex medical procedure operating location can be characterized as at least one location where a plurality of medical operators perform at least one step of a medical procedure that has a plurality of steps, where the at least one location has several (a plurality) of different operating location medical instruments that provide data to the medical operators, where the data is pertinent to the medical procedure.

As non-limiting examples, a complex medical procedure operating location can be of the type designated by a hospital or medical system as an operating room, or a catheter lab, or a hybrid operating room, or an operating clinic, or an intensive care unit, or for certain procedures an emergency room, or in some cases a mobile facility, or ambulance. As another non-limiting example, a complex medical procedure operating location can be at a veterinary or an animal facility in which the medical procedure might be performed on a non-human animal patient in order to benefit the animal or in order to perform procedures on the animal for development of improved human medical procedures.

FIG. 2A shows an example of the complex medical procedure operating locations 10 that can be associated with a complex medical procedure. At a first operating location (emergency room (ER) 12), a medical procedure such as but not limited to extracorporeal membrane oxygenation (ECMO), cardiopulmonary resuscitation (CPR), and/or ECMO-supported CPR (eCPR) can be performed. The patient can be transferred to a second operating location (Cath Lab 12), where a medical procedure such as but not limited to percutaneous coronary intervention (PCI) can be performed or a measurement can be made using fluoroscopy. The patient can be transferred to a third operating location, such as an intensive care unit (ICU) 14, prior to patient discharge 18. The period of time that a patient remains in a given operating location can differ. For example, the patient can have a medical procedure of four (4) days of ECMO in the ICU, but spend only a day or a few hours in the other operating locations.

Differing types of medical instruments and objects might be used in each different complex medical procedure operating location. For example, a different temperature probe may be used in the ER versus the ICU. However, both temperature probes are known by a medical operator to be disposed at the left nasal passage of the patient. An example system according to the principles herein can be configured to display to a display unit a plot of the temperature values from both temperature probes on the graph, with the indication of the operating location, based on the record of the example system storing medical data indicating “Instrument Identifier, Left Nasal” for each of the temperature probes.

In an example where the complex medical procedure operating location is at least one physical location in a hospital, a medical procedure may take place at several locations. In a non-limiting example, a patient may enter the hospital at the emergency room, and have an initial procedure phase diagnosing cardiac arrest with several operators and instrument measurements take place. In a second procedure phase, the patient may be moved to a catheter lab in which a stent is applied by percutaneous intervention in a second phase of the medical procedure, again using several additional instruments and operators. The patient may be moved into an intensive care unit in a third phase, in which the patient is monitored with several sensor instruments and treated with one or more pharmacological agents through drug deliver instruments until the patient discharge. From this example, it is apparent that a complex medical procedure operating location is not restricted to a single physical room or space.

FIG. 2B shows an example of an operating location logging system, according to principles of the present disclosure. The example operating location logging system provide means for collecting medical data from the medical instruments and objects used in connection with the complex medical procedure. The example operating location logging system includes one or more image recording systems 100 and a console 120. As shown in FIG. 2B, each of the example image recording systems 100 is configured to be mounted relative to a data recording object or an instrument used in the medical procedure, and is used to capture an image of a portion of the data recording object or a display of the instrument. Each example image recording system includes an image capture device 102, a mounting device 104, and a data transmission component (not shown). The mounting device 104 is configured to mount the image recording system 100 relative to a portion of a data recording object or the display of an instrument in the operating location.

As shown in FIG. 2B, the example image recording system 100 includes a linkage 106. The linkage 106 couples the mounting device 104 to the image capture device 102. The linkage 106 can be formed from any material or structure (including a telescopic or other compound structure) that is sufficiently resilient to position the image recording system 100 at a desired, targeted orientation relative to the data recording object or instrument but that also minimizes the amount of drift of the image recording system from that targeted orientation. For example, the linkage 106 can be formed from a rigid material, a semi-rigid material, a flexible material, or any combination of these materials.

The example of operating location logging system of FIG. 2B provides the capability to record and log the data events of the procedure (including data characterizing the events) that a medical operator would takes notes about using a data recording object (such as but not limited to a notebook, logbook or clipboard), without interfering with the standard methods of recording using the clipboard and logbook, and associate those event notations with other types of data from other medical instruments based on readings logged by the example systems described herein.

Some of the example instruments and objects in the operating location cannot be made to communicate data with each other. As a result, it can be difficult to get the desired information from each instrument together in a place and in a format that is useful for the medical operators.

Example operating location logging systems according to the principles herein provide example procedure programs that medical operators can use to set up a number of image recording systems to take images of, e.g., instrument displays, logbooks, and/or clipboards, and send the data to a console for recording, display, and analysis.

This is accomplished by mounting and orienting the image recording systems 100 to take images of the instrument display or the face of the clipboard or logbook, transmitting the images to the console, and allowing a medical operator to use the console 120 to select regions of interest on the display of one or more of the images (i.e., indicating the boundaries of the portion of each transmitted image that shows useful data), and allowing a medical operator to enter data to indicate operator knowledge concerning the role and import to the medical procedure of the data shown in the region of interest.

Once the operating location logging system is set up for a first patient, it does need not be re-established. Given a complex medical procedure, an operating location, and a set of image recording systems installed relative to instrument displays or objects, procedure instrument data and operator level data can be collected during an initial setup for a first patient procedure, and stored as a procedure program. The stored procedure program, including the associated operator level data, can be re-used for subsequent procedures (on the same or different patients) based on the type of the procedure program.

The example systems, methods, and apparatus described herein can be applied or installed at an operating location by one or more medical operators, including medical operators who may not participate in the medical procedure, according to the procedure program. In an example, the procedure program can be configured to cause the operating location logging system to request confirmation, such as from the one or more medical operators installing and/or operating the system, as to whether the medical data associated with the program procedure indicates the understanding and role of the data collected from the instrument display to the medical operator of the medical procedure.

In a non-limiting example, the procedure program includes the operator level data (e.g., entered at the console during a setup for a first patient) that can be reused on the same patient, or on a subsequent patient, undergoing the same specific stage(s) of a complex medical procedure. The procedure program can include at least one of the definition of the regions of interest, the region of interest identifiers, the instrument identifiers, the procedure steps, or the procedure criteria. A procedure program can include the re-usable operator level data stored in connection with the complex medical procedure, the structure of the steps of the complex medical procedure, and the structure used to specify how the re-usable operator level data is handled according to those procedure steps and procedure criteria. To the extent that the user establishes if-the-else logic to describe the procedure criteria, or instructions to the console specifying the regions of interest to record during certain of the procedure steps, this logic or instructions are also included in the procedure program to indicate the operator level data handling routines. In a non-limiting example, the procedure program could be implemented using the general programming language in which the console software is written. In another non-limiting example, a special purpose extension language could be developed to implement the procedure program. In examples according to the principles herein, patient medical data includes medical data that record the measurements, notations, and other observations for a given patient at timepoints during a complex medical procedure. Non-limiting examples of patient medical data include image data, procedure instrument data, procedure event data, medical procedure parameter(s), and any associated timestamps.

Operator level data also can be provided based on events recorded from medical operator observations during a procedure, where the medical operator makes a medical judgment to record the event as an event type. The procedure program can be recalled if a similar or related complex medical procedure is performed on another patient. If necessary, the positioning and orientation of each of the image recording systems relative to the respective instrument displays or objects, to ensure that the region(s) of interest indicated in the procedure program still substantially coincides with the portion of the image being captured using the image recording systems. The operating location logging system also can be configured to extract the data captured in the regions of interest and perform computations using the extracted data.

The example operating location logging system of FIG. 2B allows data to be gathered from many different types of instruments and objects substantially simultaneously or sequentially, and does not rely on any feature of the design or construction of any of those instruments or objects.

In some examples, the procedure program can be stored as data including indications of instrument identity, the regions of interest for each image recording system, the identity of each respective region of interest, the criteria for alerts or other notifications to be applied to extracted values, and the targets and processes associated with algorithms for a control system (such as, but not limited to, when using the instrument logging system inside a control loop to control an actuator). The data indicating the instrument identity can be, e.g., what an instrument does, or where it is attached or disposed. The data indicating the regions of interest for each image recording system can be collected using a selection tool, e.g., if an instrument display has more than one item that is to be recorded, the boundaries of the region of interest can be indicated on the image such as but not limited to using a rectangular selection boundary. The data indicating the identity of the regions of interest can include data indicating what that region of interest is named, i.e., what the regions of interest is to the medical operators.

As a non-limiting example procedure step, the example complex medical procedure can be configured such that, on starting, the blood temperature can be reduced to cause the Left Nasal sensor to read a target temperature of about 27° C.. at a rate of about 3° C./minute on the chiller, hold that temperature steady for about 12 hours, and subsequently re-warm over a period of about 12 hours to bring the reading of the Left Nasal sensor back to about 37° C..

In some examples, the data representative of the procedure step can be stored as data based on images of a clipboard or logbook recording of actions taken by medical operator during the procedure or clipboard or logbook medically-significant observations of the patient status or condition. Non-limiting examples of the procedure steps implemented by a medical operator include insertion of intravenous (IV) drip, turning on of hypothermia system, movement and re-location of patient to an intensive care unit (ICU). Non-limiting examples of the medically-significant procedure event data indicative of a patient's status or condition include data indicating the paleness of the look of the patient, starts of shivering of the patient, patient convulsion, patient degradation (including patient death), patient waking up, patient regurgitating, and patient breathing more steadily.

In example implementations, the console can be configured to reuse the medical data of a procedure program by recalling them from memory or other computer-readable media at the start of running a previously set up procedure on a new patient. Example procedure programs can be configured to be run either in a ‘recall-and-use’ mode ‘or in a ‘recall-and-edit-and use’ mode. This can simplify the setup of a complex medical procedure and make the setup for a new patient faster and more reliable.

The example operator level data of a procedure step and procedure criteria can be advantageous to integrate with procedure instrument data logging, as they can aid in the determination of what takes place in a complex medical procedure.

Example systems, methods, and apparatus described herein can be used to integrate operator level data for event types with data from instrument recordings.

Example systems, methods, and apparatus described herein allow data to be gathered from many different types of instruments and objects substantially simultaneously, and does not need to rely on any feature of the design or construction of any of those instruments or objects.

Example systems, methods, and apparatus described herein can be also provide for extraction of medical data (including medical data indicating events or steps). Extracted medical data indicating events may provide information that is not produced by an instrument display and that does not come from observations of a medical operator, but rather from applying criteria set by a medical operator to data extracted or generated at the console level.

Non-limiting examples of extraction of data and events are as follows. Given an image of an instrument display or a data recording object (such as a clipboard or logbook), data can be extracted as alphanumeric values from regions of interest by optical character recognition (OCR). Data indicating the time at which the image in the region of interest changes can be extracted from the image of the region of interest. Extracted data can be compared to procedure criteria to determine whether to trigger an alert (such as an alarm) where a value goes out of the bounds set by the criteria. This can be defined as an extracted event (such as an indication of pressure going into a danger region). Data indicating a change in a signal (e.g., where an image in a the region of interest changes) can be used as procedure criteria for determining a type of response (such as but not limited to where electrocardiogram (ECG) changes and the determination is made whether it is ST-elevation myocardial infarction (STEMI). This can be stored as an extracted event. Extracted data can be used to compute compound values (such as but not limited to a difference between left nasal temperature and right nasal temperature). Criteria and alerts can be set based on these compound values. This also can be stored as extracted events. Graphs and other plots can be generated to monitor extracted values versus time and/or compound values versus time. Complex if-then-else criteria can be generated based on plots of the extracted values and/or the compound values, and used to generate alerts or other notifications. This also can be stored as extracted events. Data from extracted values can be computed as time derivatives, or used to compute data extrapolations (e.g., temperature is rising 1° C./min), and criteria can be set for alerts on this basis (e.g., a value being predicted to exceed 37 in 6 minutes can be used to trigger an alarm for intervention prior to the predicted value being reached). This can be of clinical value, since the ability to intervene before an undesirable threshold is reached can be advantageous.

Using example systems, methods, and apparatus described herein, data not present on or extractable from instrument displays can be computed at the console based on medical operator instructions and/or medical operator set criteria. This can be used to create valuable data and useful warnings to improve the medical procedures.

In some examples, the operating location logging system can be used to compute at least one medical procedure parameter using one or more of the differing types of medical data (including the procedure instrument data, and the procedure event data), and display the results on a display of the console. For example, the medical procedure parameter can be displayed to a user on the console display as a graphical representation of the at least one medical procedure parameter graphs, plots and other navigable graphical representation. The operating location logging system allows an analysis of the activities, events, and procedures in connection with the medical procedure to be made available to a user in a single central location.

In some examples, the operating location logging system can be used to compute at least one alert parameter that corresponds to the medical event, and generate at least one graphical representation of the alert parameter (graphs, plots and other navigable graphical representation). Operator level data indicating what value or change of the procedure instrument data or the procedure event data could be cause for an alert can be used for computing the alert parameter. For example, the medical data can indicate that the temperature derived from image data for a first region of interest of a first image should not be allowed to exceed about 37° C.. (i.e., issue an alarm warning if it does), and/or issue an alarm warning if the MAP pressure derived from image data for a second region of interest of a second image is falling faster than 1 mmHg/minute. As another example, the medical data can indicate that an alert should be issued if a portion of a clipboard is updated to indicate a notation or check-mark that an infusion pump syringe is empty (e.g., a box is checked), as the medical operator refills it.

Once the image recording systems are mounted, the regions of interest are indicated for each image captured using the image recording systems, and other types of operator level data is established for each object or instrument for a first performance of a given medical procedure, the information and data may be stored as a procedure program for the medical procedure. The procedure program for a given medical procedure can be later retrieved, based on a user request, for the second, third, and other subsequent performance of the same given medical procedure on other patients. In particular, the retrieval of the stored medical procedure program causes the operating location logging system to establish the previously-indicated regions of interest to be monitored for each image captured based on where and how the image recording system is mounted, and to use the definitions previously entered for other types of operator level data for each object or instrument device to monitor the medical procedure and perform the analyses and computations established from the first performance of the medical procedure.

In an example, the linkage 106 of the example operating location logging system of FIG. 2B can be formed from a flexible material or structure that is sufficiently resilient to position the image recording system 100 at a desired, targeted orientation relative to the data recording object or instrument such that the amount of drift of the image capture system from that targeted orientation is minimized over a specified period of time (T). The time scale T can be on the order of the length of time of the medical procedure, or the length of time that the particular object or instrument is used in the medical procedure, or T can be long enough to be on the order of several different medical procedures. For example, the time scale T can be on the order of hours to days. In addition, the image capture device 102 has a principal dimension in a measure of size (such as diameter, length, or width) such that it causes little to no (i.e., minimal) obstruction of the view of the data recording object or the display of the instrument when the image recording system 100 is positioned at the target orientation.

The image recording system can be configured to capture the images at predetermined time intervals. For example, for an instrument that refreshes the display at specified time intervals, such as but not limited to a cardiac monitor, the image recording system can be configured to capture the images at time intervals that correspond to the refresh rate. In another example, the image recording system can be configured to capture the images based on motion detection or a field-of-view intrusion. The image recording system includes at least one transmitting unit to transmit the image data for the captured images to a console. The image data can include a timestamp associated with the raw images. The transmitting unit can be configured to transmit the image data via a wired means or a wireless means. As non-limiting examples, the image data can be transmitted using a USB device, Ethernet, Bluetooth, WIFI, or other means.

In a non-limiting example, the image capture device 102 can be a small camera. The example camera can be positioned so that it can capture one or more images of the display of the instrument or the data recording object, and is of a size (principal dimension) that does not significantly block a user's view of the data recording object or instrument display. For example, the principal dimension can be about 2 inches or less, about 1 inch or less, or about 0.5 inch. The example camera can be of any shape, including cylindrical, spherical, hemispherical, cuboid, rectangular, or other polygonal shape. In the targeted position, the camera is selected to be of a size that does not significantly obstruct the view of a user during performance of a medical procedure.

An example mounting device according to the principles herein can be a clamping means that allows the image recording system to be coupled relative to the data recording object or instrument whose display is to be recorded. The example mounting device is configured to be stable over the duration of time of the medical procedure.

In a non-limiting example, the linkage is configured such that a user may attach the mounting device (such as but not limited to a clamp) relative to an instrument and object, and bend the linkage so that the image recording system is correctly positioned to take the desired image of the data recording object or the display of the instrument. The linkage is formed from a material or structure that is sufficiently stiff so the position of the image recording system exhibits minimal drift (or less drift than an acceptable threshold limit) over the duration of the medical procedure.

FIG. 2C shows a non-limiting example console and example medical instruments mounted to a mobile unit 200, according to principles of the present disclosure.

The example console includes the display unit 124 and a user input device 202 (such as but not limited to a keyboard). The example medical instruments include a chiller module 204 and an uninterruptible power source (UPS) 206. The example chiller module 204 can provide reliable dual-channel heater/chillers support for both ECMO and CSDH loops. The example UPS 206 is a power source that can support the console and/or at least one ECMO pump, to allow the ECMO and hypothermia procedures during transport of a patient from the ER to the Cath/Operating Room to the ICU.

FIG. 3 shows a block diagram of an example operating location logging system 300, according to principles of the present disclosure. The example operating location logging system includes a non-limiting example console 300 and at least one image recording system 320. The example console 300 includes at least one memory 302 and at least one processing device 122. The at least one memory 302 is configured to store processor-executable instructions 306, an analyzer module 308, and data 310. As described in greater detail below, data 310 can include one or more of procedure instrument data, procedure event data, and operator level data. The at least one processing device 122 is communicatively coupled to at least one image recording system 320 and the at least one memory 302. The image recording system 320 includes a data transmission component 322. The data transmission component 322 is used to transmit image data representative of images captured by the image recording system 320 to the console 300 via wireless or wired communication, or other means. The analyzer 308 can be used to perform any of the computations or to generate any of the graphical representations described herein.

The example processing device 122 can be programmed to perform any of the methodologies and processes described herein. In these examples, the processing device 122 can be configured to execute the processor-executable instructions 306 stored in the memory 302 to receive image data transmitted using the data transmission component 322, where the image data corresponds to the one or more images captured by the image recording system, and associate a timestamp with the image data. The processing device 122 can cause the example analyzer 308 to analyze the image data to determine the occurrence of a medical event in connection with the medical procedure, based on procedure instrument data and/or procedure event data associated with the medical procedure. The processing device 122 can be configured to execute the processor-executable instructions 306 stored in the memory 302 to associate operator level data with region(s) of interest of the one or more images, associate the medical event with the at least one timestamp of the corresponding region of interest, and store the image data associated with the at least one region of interest, the medical data, and the associated timestamp in a database. In an example, the medical data can be the indication of the boundaries of at least one region of interest made by a user relative to the image. In response to a user request, retrieve from the database the previously stored image data associated with the region(s) of interest and/or the previously stored operator level data.

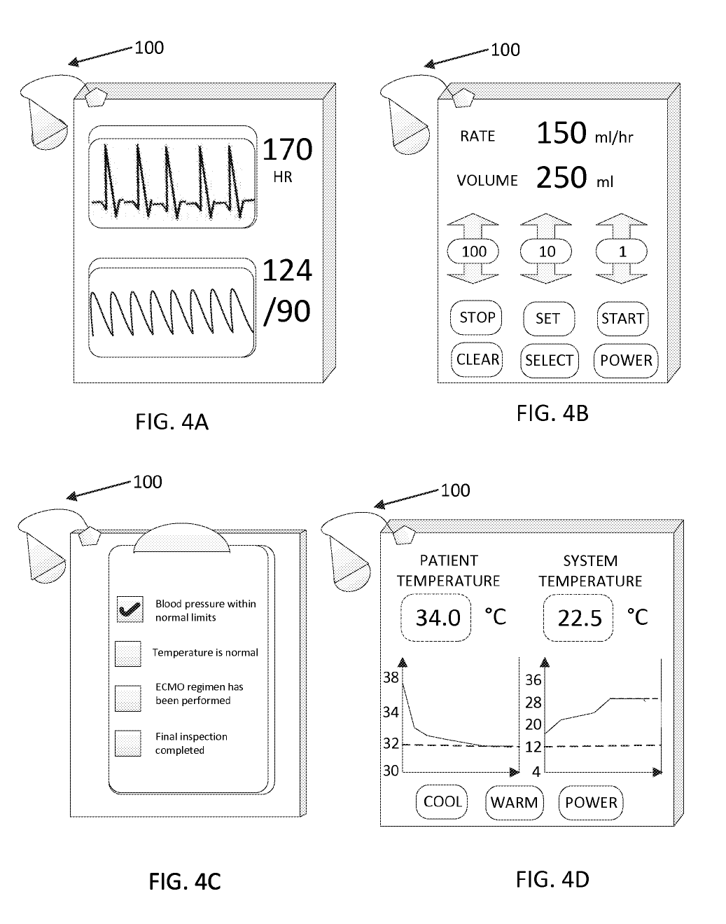

FIGS. 4A-4D shows non-limiting examples of image recording systems mounted relative to objects or instruments used in a medical procedure, for capturing images. In various examples, the instrument or object can be any item present in or coupled to a laboratory, a medical procedure, an intensive care unit, an emergency room, or other facility where a medical procedure is performed. In any example, the image can be of a portion of a data recording object, such as but not limited to a clipboard or a logbook being used during the medical procedure. For example, the example image can show where a checkbox on a clipboard has been checked, or other information is written to the logbook or on the clipboard.

FIG. 4A shows an example image recording system 100 mounted relative to a display of an instrument. In this example, the instrument is a heart monitor. Analysis of image data from images of the display of the heart monitor can be used to provide procedure instrument data such as but not limited to the value of heart rate, pulse, and value of blood pressure.

FIG. 4B shows an example image recording system 100 mounted relative to a display of another instrument. In this example, the instrument is an infusion pump. Analysis of image data from images of the display of the infusion pump can be used to provide procedure instrument data such as but not limited to the value and units of the rate of infusion, the units of the rate, and the volume of substance infused.

FIG. 4C shows an example image recording system 100 mounted relative to a data recording object. In this example, the data recording object is a clipboard. Analysis of image data from images of the clipboard can be used to provide procedure event data indicating where a checkbox on the clipboard has been checked, or other information is hand-written to the clipboard. The procedure event data can be determined based on hand-writing analysis of notations made to the clipboard. In non-limiting examples, the capture rate of the image recording system can be set to regular intervals, e.g., to a value ranging from at least one image every 10 seconds to at least one image every two minutes. For example, the capture rate can be at least one image every 30 seconds or every 60 seconds. The example operating location logging system can be configured to identify any differences between the captured images of the data recording object to determine if any features have changed, e.g., indicating that a checkmark or hand-written notation has been made to the data recording object. The associated timestamp for the captured image can be associated with the operator level data obtained from the image analysis.

FIG. 4D shows an example image recording system 100 mounted relative to a display of another instrument. In this example, the instrument is a monitor for a hypothermia system. Analysis of image data from images of the display of the monitor can be used to provide procedure instrument data such as but not limited to the value and units of the patient temperature measurement and the value and units of the system temperature measurement.

An example console according to the principles herein can include a display configured to receive user input relative to the images displayed. In an example, the images can be displayed along with the associated timestamp. The example console is configured to display a rendered interactive user interface element that allows a user to define at least one region of interest of each image displayed. Non-limiting examples of the interactive user interface element includes a cursor or a selection tool. Each of the selected regions of interest is associated with the timestamp relevant to the respective image. The example console is also configured to display a rendered interactive user interface element that allows a user to enter the procedure level data associated with each of the selected region of interest of the image. In an example, the procedure level data entered for each region of interest can identify the data type, identify the association of the kind of data and what role that data plays in the procedure, such as but not limited to what the data means to stage or status of the medical procedure.

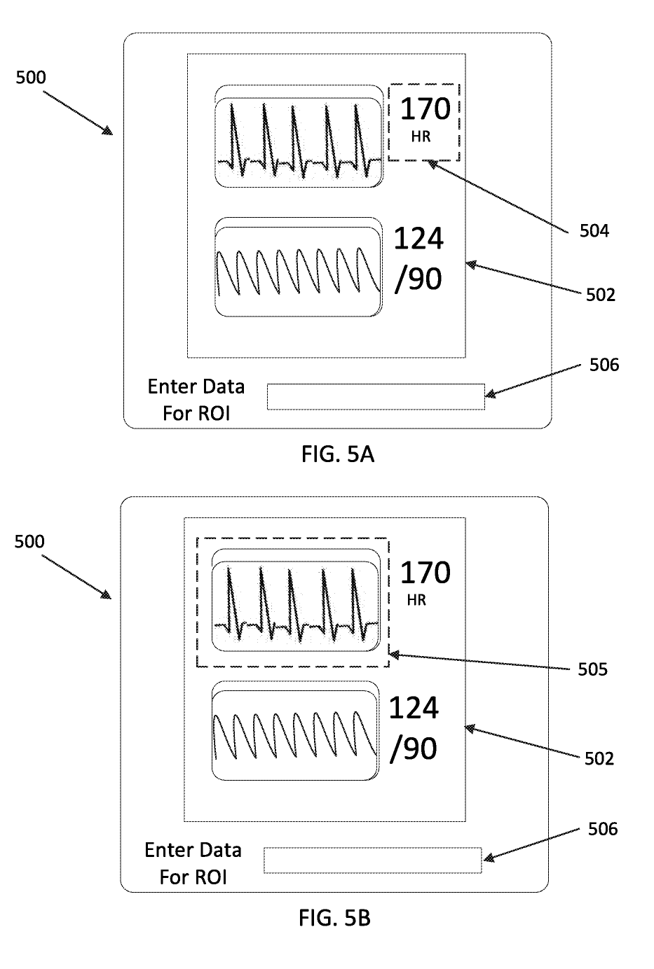

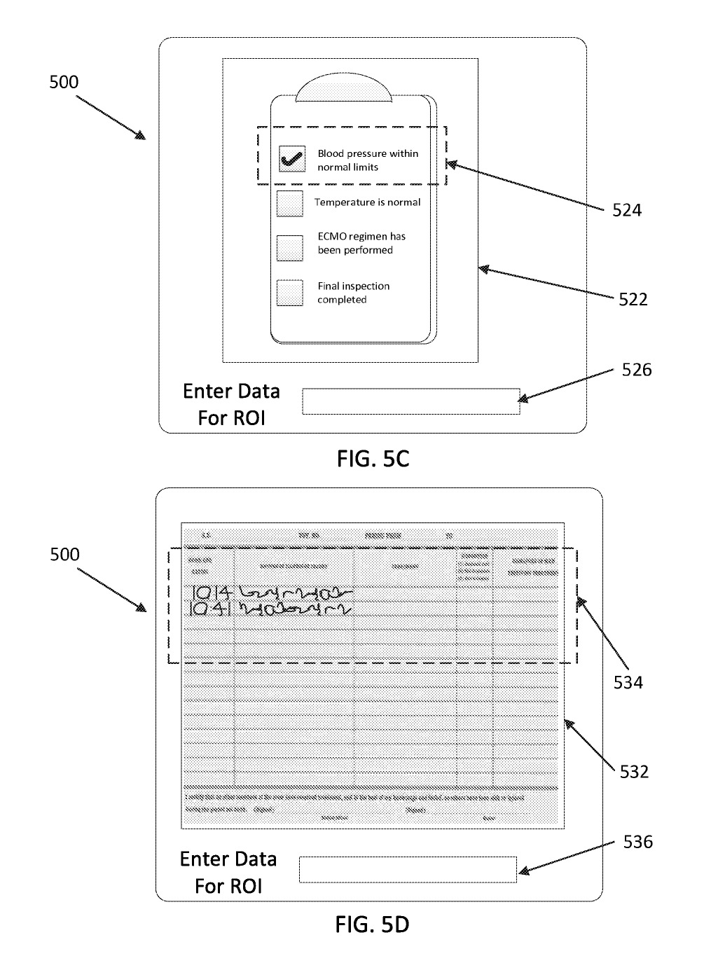

FIGS. 5A-5D show screens of example consoles of an operating location logging system, according to principles of the present disclosure.

FIG. 5A shows an example of use of a console display 500 to show an image of a heart monitor display 502, the boundaries of a first region of interest 504 indicated by a user, and an interactive user interface element 506 that is rendered to allow a user to input other operator level data pertaining to the first region of interest 504 as described herein. FIG. 5B shows the example console display 500 with the image of the heart monitor display 502, with the boundaries of a second region of interest 505 indicated by a user at a differing portion of the same image 502, and the rendered interactive user interface element 506 that allows the user to input other operator level data pertaining to the second region of interest 505 as described herein.

FIG. 5C shows the example console display 500 with the image of a clipboard 522, with the boundaries of a region of interest 524 indicated by a user as a portion of the image 522, and a rendered interactive user interface element 526 that allows the user to input other operator level data pertaining to the region of interest 524 as described herein. Procedure event data can be derived based on an analysis of the image data pertaining to the region of interest 524, such as where a check-mark is made to the clipboard or from hand-writing analysis.

FIG. 5D shows the example console display 500 with the image of a logbook 532, with the boundaries of a region of interest 534 indicated by a user as a portion of the image 532, and a rendered interactive user interface element 536 that allows the user to input other operator level data pertaining to the region of interest 534 as described herein. Procedure event data can be derived based on an analysis of the image data pertaining to the region of interest 534, such as hand-writing analysis of notations made to the logbook.

At least one processing unit of the example operating location logging system can be programmed to extract procedure instrument data or procedure event data corresponding to each of the defined regions of interest. The procedure instrument data or procedure event data can include one or more of: alphanumeric data, a graphical feature associated with an image object shown in the region of interest, or a notation made in portion of the image shown in the region of interest. The at least one processing unit of the example system is also programmed to associate the extracted procedure instrument data or procedure event data with the timestamp of the image based on which the region of interest is defined. In some examples, the extraction can be performed to determine values of changes in the timestamps associated with the regions of interest as either procedure instrument data or procedure event data. That extraction operation may be performed using the at least one processing unit. As a non-limiting example, the extraction can be based on application of an optical character recognition technique to the region of interest, to extract alpha-numeric data from the images, such as but not limited to data indicative of measures of pressures, temperatures, flow rate, quantity of substance administered, or other such measure. The extracted alpha-numeric data can also include text data indicative of units, filenames, etc. The extraction process can derive data indicative of feature recognition, change in timestamp, image feature recognition, or changes of the face of a clipboard. The feature recognition can include data indicative of ST elevation in ECG traces. The change in timestamps can be used to recognize when an image changes in a significant way from a first timestamp to another timestamp. The image feature recognition can be for 2D images captures such as ultrasound, angiography, fluoroscopy, OCT, or other images. For example, the extracted image feature recognition can be used to indicate if a measure of the arterial diameter shown in the image(s) change. Non-limiting examples of the changes to a clipboard that can be imaged include changes made to the checkboxes on the clipboard which indicate pre-specified event or other types of events (including unanticipated events) that might take place during or after the medical procedure. The data extraction process may include applying text recognition or handwriting recognition tools to the image of the region of interest of the clipboard. The data extraction process may include applying means for recognizing values of settings of a dial, vernier, or other analog display component or digital equivalents.

In examples, the one or more processing units can be co-located with at least one of the image recording system, the console, or at a remote location (such as but not limited to a server in the cloud). In a non-limiting example, the extraction of data indicative of a region of interest may be performed using a processing unit co-located with the IRS. This allows the IRS to send extracted values rather than whole images. In another non-limiting example, the example at least one processing unit may be located at a remote location, such that the console can send the images from the regions of interest to the remote location and gets back extracted data for each timestamp associated with the image(s).

In an example, the console can be configured to display a rendered representation of the extracted data along with the image of the region of interest, as well as a rendered interactive user interface element that seeks confirmation from a user that the extracted data is accurate.

The at least one processing unit also can be programmed to compute one or more medical procedure parameters using the medical data, and the associated timestamp, and generate at least one graphical representation of the one or more medical procedure parameters. For example, the graphical representation can be a graph of continuous variables computed based on the extracted data, such as but not limited to pressure, flow rate, temperature, heart rate, blood oxygen, blood gas levels, or others parameters of interest to one of ordinary skill in the art. The graphical representation can show icons (such as but not limited to tick marks) to a timeline in a graph along an to indicate the evolution of a parameter over time on a graph. In an example, there can be indicators placed on the graphical representation to show a timestamp for an event occurring during the medical procedures, that correlates to the occurrence of a notation being made on a clipboard or in a logbook. In another example, there can be indicators placed on the graphical representation to correspond to a timestamp where a region of interest on an image changes above or below a predetermined threshold, or a feature in an image changes.

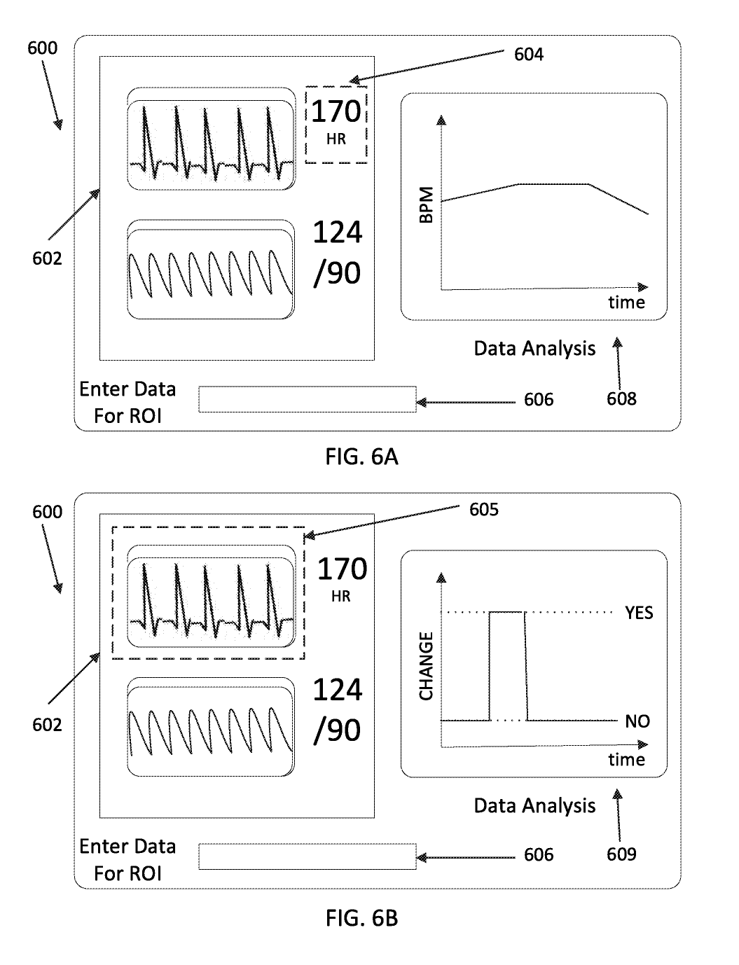

FIGS. 6A-6B show screens of another example console of an operating location logging system, according to principles of the present disclosure. In these examples, the display is used to show a rendering of the at least one graphical representation of the at least one medical procedure parameter computed using the medical data and the associated timestamp. For example, FIG. 6A shows an example use of a console display 600 to show an image of a heart monitor display 602, the boundaries of a first region of interest 604 indicated by a user, a rendered interactive user interface element 606 to allow a user to input other operator level data pertaining to the first region of interest 604, and a first graphical representation 608 of the medical procedure parameter computed based on the medical data extracted from image data pertaining to the first region of interest 604. In this example, the medical procedure parameter is a measure of heart rate and the graphical representation 608 is a plot of values of the heart rate over time. FIG. 6B shows another example use of console display 600 with the image of the heart monitor display 602, with the boundaries of a second region of interest 605 indicated by a user at a differing portion of the same image 602, the rendered interactive user interface element 606 that allows the user to input other operator level data pertaining to the second region of interest 605, and a second graphical representation 609 of the medical procedure parameter computed based on the medical data extracted from image data pertaining to the second region of interest 605. In this example, the medical procedure parameter is the shape of each scans and the graphical representation 609 is a plot showing whether the shape of the scans changes at a given time point during the procedure.

The at least one processing unit can be further programmed to compute at least one alert parameter that corresponds to the at least one medical procedure parameter, and generate at least one graphical representation of the at least one alert parameter.

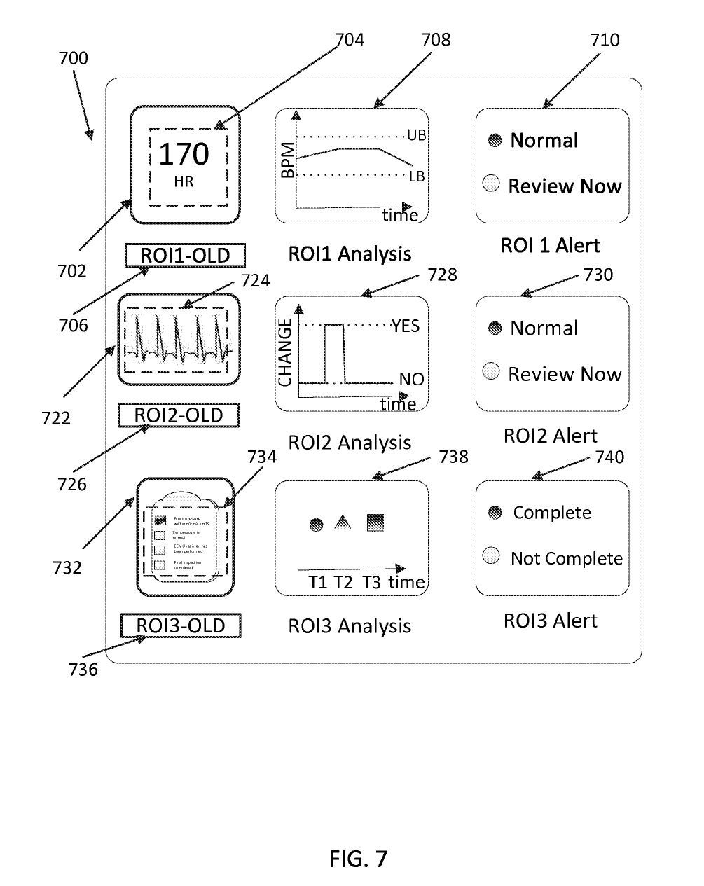

FIG. 7 shows a screen of another example console of an operating location logging system, in which the results of the analyses of multiple instruments and objects are shown. In these examples, the display is used to show a rendering of the at least one graphical representation of the at least one medical procedure parameter, as well as alert parameters, computed using the medical data and the associated timestamp. FIG. 7 shows an example use of a console display 700 to show an image of a portion of a heart monitor display 702, the boundaries of a first region of interest 704 indicated by a user, a rendered interactive user interface element 706 to allow a user to input other operator level data (OLD) pertaining to the first region of interest 704, and graphical representations 708, 710 of the medical procedure parameter (708) and the alert parameter (710) computed based on the medical data extracted from image data pertaining to the first region of interest 704. In this example, the medical procedure parameter is a measure of heart rate, the graphical representation 708 is a plot of values of the heart rate over time, and also shows dashed lines indicating the procedure criteria of values of an upper bound (UB) and a lower bound (LB) of the values of heart rate. The graphical representation 710 is an alert parameter based on the heart rate. FIG. 7 also shows an image of a different portion of the heart monitor display 722, the boundaries of a second region of interest 724 indicated by a user, a rendered interactive user interface element 726 to allow a user to input other operator level data (OLD) pertaining to the second region of interest 724, and graphical representations 728, 730 of the medical procedure parameter (728) and the alert parameter (730) computed based on the medical data extracted from image data pertaining to the second region of interest 724. In this example, the medical procedure parameter is the shape of the scans, the graphical representation 728 is a plot showing whether the shape of the scans changes at a given time point during the procedure, the graphical representation 730 is an alert parameter based on the shape of the scans. FIG. 7 also shows an image of a clipboard 732, with the boundaries of a third region of interest 734 indicated by a user as a portion of the image 732, a rendered interactive user interface element 736 that allows the user to input other operator level data (OLD) pertaining to the third region of interest 734, and graphical representations 738, 740 of the medical procedure parameter (738) and the alert parameter (740) computed based on the medical data extracted from image data pertaining to the third region of interest 734. Examples of the procedure event data include a check-mark being made to the clipboard or hand-written notation being made. The graphical representation 738 can be a timeline of active markers, showing when events occurred during the procedure, where a user activation of each marker can cause the system to display or otherwise provide data and other information concerning the activity and occurrence connected with the event. In the example of FIG. 7, the active markers are shown with differing shapes, each indicating that a different set of activities are associated with the procedure event data corresponding to each shape. The example alert can be based on indications of whether a specified task for a procedure is completed or not.

In a non-limiting example, the timeline display of timestamped events from a data recording object, such as but not limited to a clipboard or other procedure event data logging element, can include discrete indicators (including tick marks) on a timeline displayed to the console. In some examples, when the user selects a specific discrete indicator associated with a timestamp on the timeline, a record of the original region of interest associated with the timestamp may be displayed to the user, allowing recall and inspection of relevant handwritten notes (even without any handwriting analysis or other extraction being performed).

As described above, the at least one processing unit also can be configured to generate graphical representations based on the extracted data from more than one region of interest in an image, or more than one regions of interest in multiple images. In another example, the graphical representation can be of data for two or more variables, where the data for each variable is derived from an extraction from two of more different regions of interest in one or more image(s). The graphical representation can be computed based on composite (i.e., compilations of) variables from different regions of interest, such as but not limited to differences between values of pressures at a time T1 as a percentage a value computed for a time T2.

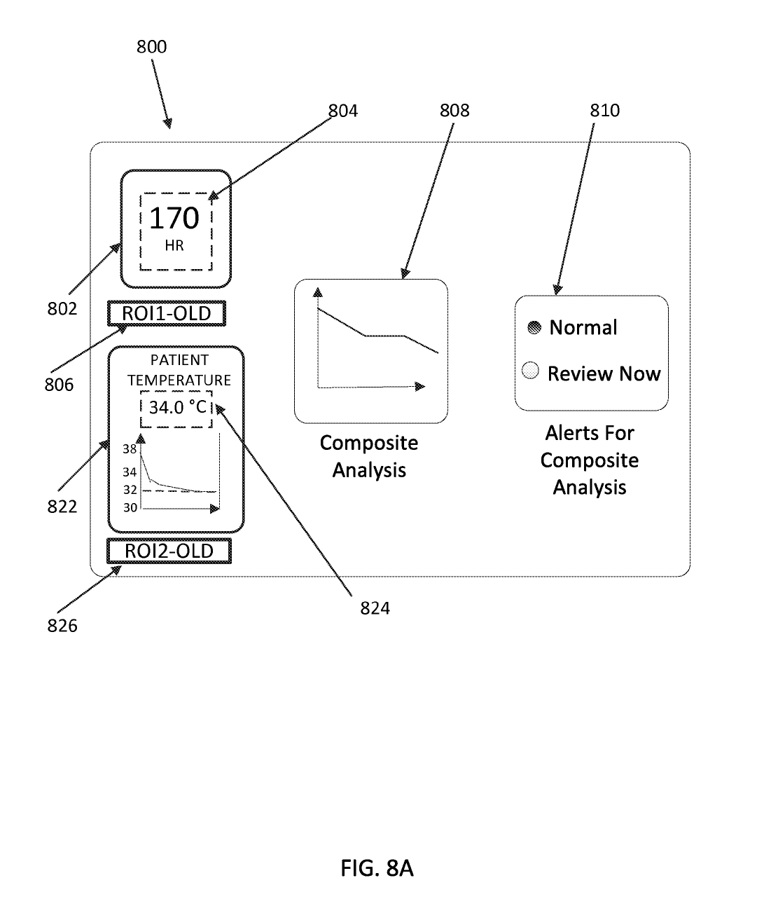

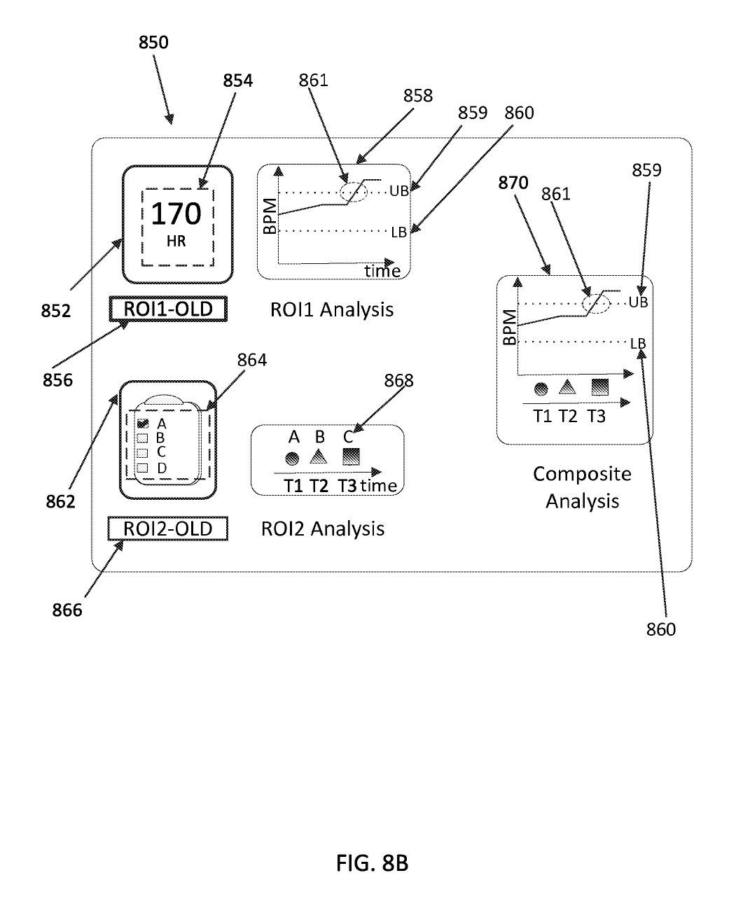

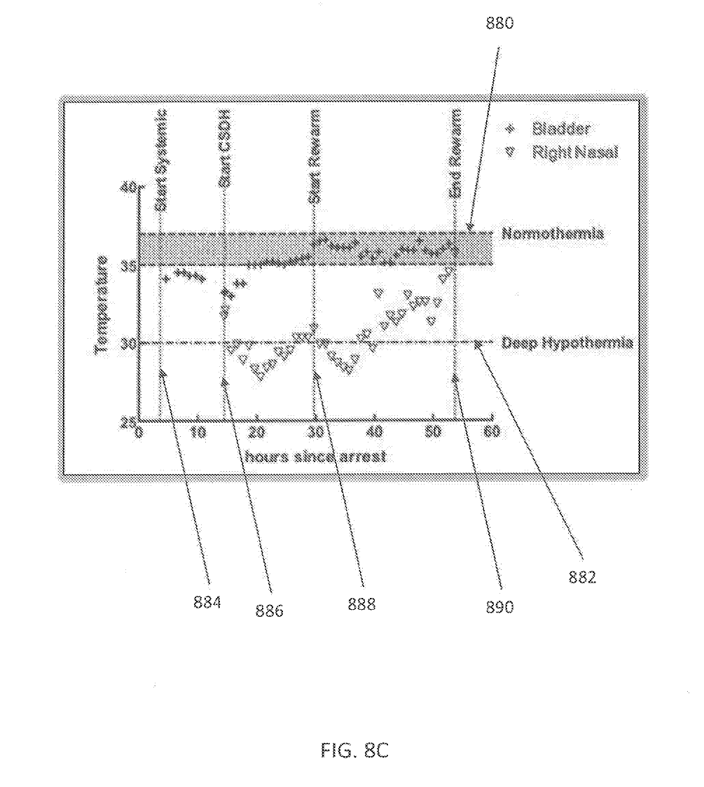

FIG. 8A shows a screen of another example console of an operating location logging system, in which the results of the composite analyses of multiple instruments and objects is shown. In this examples, the display is used to show a rendering of the at least one graphical representation of the at least one medical procedure parameter, as well as alert parameters, computed using the medical data and the associated timestamp from two or more differing regions of interest. FIG. 8A shows an example use of a console display 800 to show an image of a portion of a heart monitor display 802, the boundaries of a first region of interest 804 indicated by a user, a rendered interactive user interface element 806 to allow a user to input other operator level data (OLD) pertaining to the first region of interest 804, an image of a portion of a hypothermia monitor display 822, the boundaries of a second region of interest 824 indicated by a user, and a rendered interactive user interface element 826 to allow a user to input other operator level data (OLD) pertaining to the first region of interest 824. A composite graphical representation 808 of a medical procedure parameter is shown, based on a computation of the medical data extracted from image data pertaining to the first and second regions of interest 804, 824. The composite graphical representation 810 of the alert parameter is also computed based on a computation of the medical data extracted from image data pertaining to the first and second regions of interest 804, 824.

FIG. 8B shows an example use of a console display 850 to show an image of a portion of a heart monitor display 852, the boundaries of a first region of interest 854 indicated by a user, a rendered interactive user interface element 856 to allow a user to input other operator level data (OLD) pertaining to the first region of interest 854, and a graphical representation 858 of the medical procedure parameter. In this example, the medical procedure parameter is a measure of heart rate, and the graphical representation 858 is a plot of values of the heart rate over time. FIG. 8B also shows dashed lines indicating the procedure criteria of values of an upper bound (UB) 859 and a lower bound (LB) 860 of the values of heart rate. In an example, the system can be configured to issue an alert if a threshold set as a procedure criterion is exceeded, such as at area 861 of the plot where the value of BPM crosses upper bound (UB) 859. As a result of the violation of the procedure criterion and/or triggering of the alert, the example operating room logging system can be configured to determine associated the procedure instrument data and/or procedure event data, such as but not limited to using data analysis (including using extraction),nd/or other types of analysis. FIG. 8B also shows an image of a clipboard 862, with the boundaries of a second region of interest 864 indicated by a user as a portion of the image 862, a rendered interactive user interface element 866 that allows the user to input other operator level data (OLD) pertaining to the second region of interest 864, and a graphical representation 868 of the medical procedure parameter. Examples of the procedure event data include a check-mark being made to the clipboard or hand-written notation being made. The example graphical representation 868 is a timeline of active markers, showing when events occurred during the procedure, where a user activation of each active marker can cause the system to display or otherwise provide data and other information concerning the activity and occurrence connected with the event. In the example of FIG. 8B, the active markers are shown with differing shapes, each indicating that a different set of activities are associated with the procedure event data corresponding to each shape. The composite graphical representation 870 shows the composite analysis of the medical procedure parameter and the active markers, ordered versus time, to provide a user with an indication of the procedure event data (via the active markers) that correspond to values and time variation of the medical procedure parameter. For example, user activation of each active marker can cause the system to display or otherwise provide data and other information concerning the activity and occurrence connected with the procedure event data at each marker, including the details of the check-mark or hand-written notation made to the data recording object (such as but not limited to the clipboard). In the example of FIG. 8B, user activation of the active marker at time T3 could cause the console to display the procedure event data corresponding to the time that the values of the heart rate exceed the procedure criteria set as the upper bound (UB) 859, including displaying the image that shows the check-mark or activity description or hand-written notation that corresponds to the check-mark.

FIG. 8C shows an example graphical representation as a plot of temperature measurements versus time since cardiac arrest for a patient, according to principles of the present disclosure. The graphical representation includes procedure criteria for the medical procedure, with the procedure criterion at 880 being a threshold value for normothermia and the procedure criterion at 882 being a threshold value for deep hypothermia. FIG. 8C also shows dotted lines corresponding to procedure steps 884, 886, 888, 890.

The example console can be configured to display the graphical representations of the variables from a region of interest or composite variables as described hereinabove in relation to values (or a band or range of values). For example, the extracted pressure data can be shown in a graphical representation including an upper bound indicating a user-specified high-pressure value and a lower bound indicating a user-specified low-pressure value, thereby establishing a pressure band that the medical operator sets to generate high and low pressure alerts. The values of the high-pressure and low-pressure can be received as procedure level data described hereinabove.

The example console can be configured to display the graphical representations with event flags to indicate the relationship between continuous time variables and procedure events.

In examples of the operating location logging system, the graphical representation of the medical procedure parameters can include, for example, patient condition status icons, numbers, gauges, bar graphs, pie charts, lines, points, heat maps, Harvey balls, arrows, emoticons, colors, gradient coloration of icons, completeness of icons, size of icons, etc. The graphical representations can also include, for example, a modification of a virtual representation of a physical object (e.g. changing the color, size, or shape, of a data recording object or section within the region of interest of the image based on a metric). In examples of the operating location logging system, the graphical representations could be represented as floating graphs in particular areas of the display of the at least one region of interest of the image. The graphical representations can also include avatars, or a representation of the image including a portion having a different size or color to indicate the region of interest. The graphical representation of the medical procedure parameter could be morphed to represent changes in the procedure level data, the procedure instrument data or procedure event data, or a boundary of the region of interest. The graphical representations, procedure level data, and procedure instrument data or procedure event data used to generate the graphical representations described herein can change based on the type of review or the level of granularity of the review.

In an example, a system, method and apparatus is provided for logging the procedures and activities in an operating location, based on timestamps, by recording face of the instruments and objects, and obtaining operator level data, procedure instrument data, .and procedure event data.

In an example, a system, method and apparatus is provided for performing a primary extraction to set new operator level data. For example, a medical operator can get a sense of where things are relative to upper and lower bounds set for parameters and values being extracted using the medical data. At this level, a medical operator can add complex statements to set alert parameters, such as a rule that if a certain medical instrument value is within set bounds (i.e., set values of upper and lower bounds), the system informs the medical operator if other medical instruments are out of their respective set bounds.

In an example, a medical event can be defined as occurring if the measurements of an instrument, or a computation based on two or more instrument values, goes out of the set values of upper and lower bounds, such as could be set with an alert. If procedure instrument data and procedure event data is extracted from a plurality of instruments and objects, the system is configured to evaluate the change of an instrument value going out of bounds as an indication of a medical event. Given operator knowledge stored as procedure criteria specifying an upper bound and a lower bound of an instrument measurement, notification of the measurement going out of bounds can be classified as a medically-significant event. Operator specifies what levels are safe, which levels might be guarded, and what values indicate danger. The graphs can show notifiable events about things that change over time, such as rate of change of pressure, the difference between sensor readings, these are example criteria for setting of notifiable events. Extrapolations of graphs can be used as predictive tools for potential medical events that may occur.

For a clinical protocol, it is difficult to specify the frequency of information gathering around the operating location. For example, it is difficult to specify that an individual is to check these values of these instruments in a specific sequence at a regular time interval (e.g., every 5 minutes). In practice, this is done as often as an assigned surgical assistant is able to make the round to check each piece of equipment. Important data may never be gathered, e.g., where an assistant is not able to make the rounds at a time when data from an instrument could have provided advances notice of a condition going downhill, potentially leading to a medical event. As another example, the data from an instrument may not be collected fast enough manually so that an individual could tell how noisy the data is.

In addition to coordinating the receipt and analysis of data from so many varied types of instrument displays, the operating location logging system allows indications using checkboxes and writings in logbooks (e.g., notes on events in procedure such as “doctor inserted stent at time t.”), to become manipulable data. Each is collected and given machine-accurate timestamp, as a way to move quickly from paper methods to electronic format.

In an example implementation, images from human logging can be recognized, e.g., using a checkmark, and can be used to set off other notifications. The checkboxes mean various things—the names and other words next to the checkboxes can be used to indicate the role of the data to the medical procedure.

In an example, systems, methods, and apparatus are provided herein that allow for using the example logging system in the operating location to extract instruments display data together with the data recording object (i.e., logbook/clipboard) data.

In an example, systems, methods, and apparatus are provided herein that allow for timestamping of the procedure instrument data and the procedure event data brought together, and allow data extraction and user notification to be provided at a single point (i.e., a console).

In an example, systems, methods, and apparatus are provided herein that allow for graphs and other computations to be exhibited across many different timepoints.

Various types of operator level data is useful to the example operating location logging systems herein. For example, if there are three different pressure sensors, the operator level data gives data distinguishing all three sensors. In an example, the different air conditioner settings in an operating location might affect some of the sensor data measurements. The operating location logging system could be used to point out inconsistencies or discrepancies in the instrument measurements.

A complex medical procedure might be repeated on different patients multiple times, or a patient might come in several times per month for the same procedure. The same camera might be hooked up to different instruments, so an assistant may need to re-check the setup of the operating location logging system and settings occasionally before the start of a repeated procedure.

The data can show how rapidly events of an operation takes place, e.g., insertion of a stent may take place in a few minutes.

The settings are meant to pertain to a type of procedure. That is, when the setup is done using the operating location logging system, it is performed seamlessly as would be performed any other medical procedure. In setting up a team and rehearsing a procedure, medical practitioners verify and agree about what is desired to be monitor during the medical procedure. The example operating location logging system can be configured to monitor the desired instruments and objects, according to the frequency set by the medical practitioners, and data analysis can be performed in real-time as described according to the principles herein.

With an example system herein implemented, the individual who normally does the manual logging and circles the operating location noting instrument values can now spend more time doing review of occurrences and can make notes with greater detail about what is going on in the operating location.

Optical character recognition can be more reliable than notations made based on observations of an individual in a logbook (e.g., where an individual may make digit transposition errors). For semi-automated extraction, the example system could be configured to prompt an individual occasionally or regularly to review and confirm the accuracy of the values of the extracted data. For manual extraction, an individual could enter as user input numbers or other info (data) in fields on the graphical user interface on the console display, relative to the regions of interest. For fully automated extraction, the example system could be configured to perform the operations described herein in the absence of use input.

The example operating location logging system can be configured for data extraction from multiple differing instruments (i.e., machines) and objects. The example system is applicable to a plurality of different instruments, from several different manufacturers, with at least one instrument being unable to send or receive data, and each instrument not being able or configurable to communicate with each other.

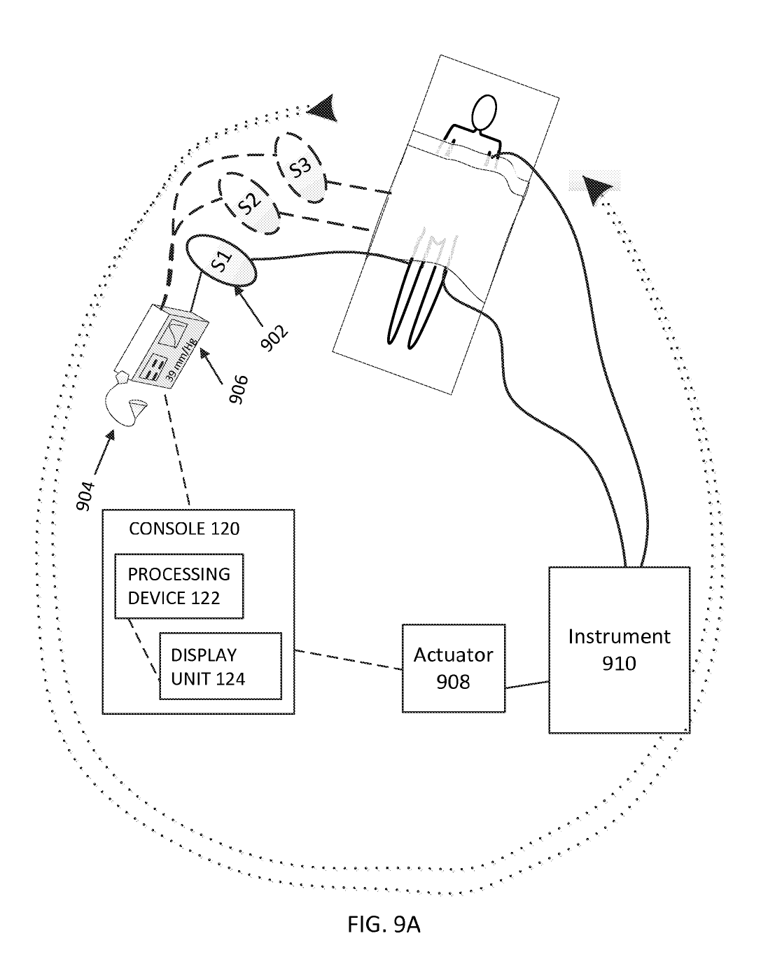

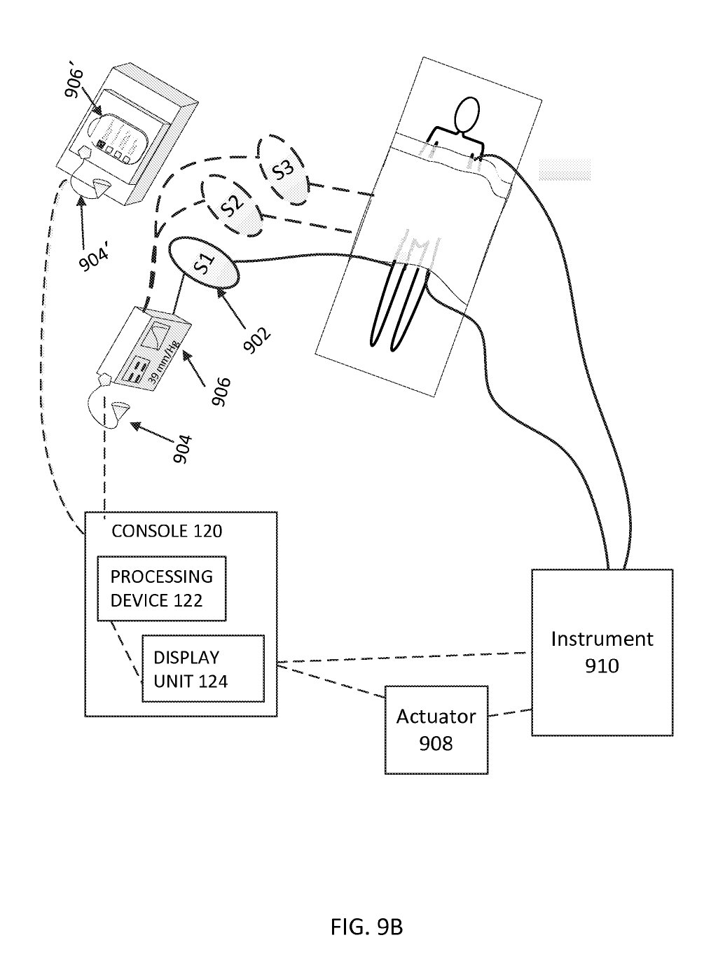

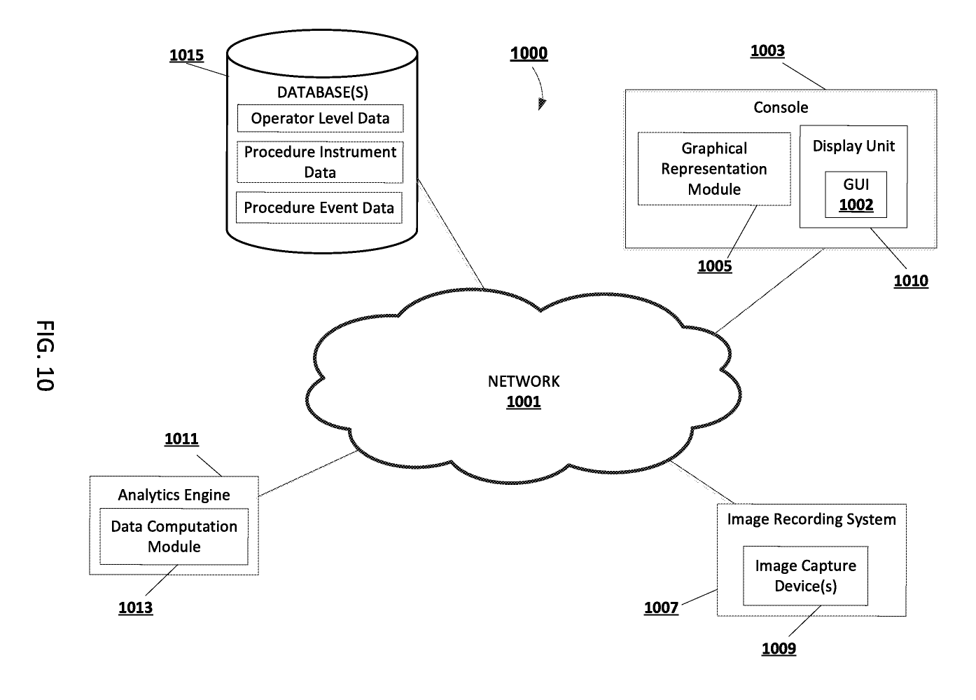

FIG. 9A shows an example control system that includes a sense medical instrument (sensor Si 902), an image recording system 904 to capture images of instrument display 906 for display of sensor data, an actuator 908, and a drive medical instrument. For example, the system could take data from a sensor 902 in the bladder and run an instrument 910 (such as but not limited to a heater/chiller coupled to a water blanket wrapped around the patient). The example logging system is part of a loop in the control system for the heater/chiller. An example implementation of the control system includes performing sensor measurements using the sensor Si 902, using the image capture device to capture images of the instrument display 906 for the sensor S1 902, and using the data transmission component to transmit the image data to the console 120. The processing device 122 of the console 120 is used to perform the image analysis (such as data extraction) to provide procedure instrument data, and using the procedure instrument data from the data analysis to control the actuator 908. The actuator 908 is used to control the heater/chiller 910, such as but not limited to controlling the operating setting if the example system determines based on the analysis that the sensor measurement is to be adjusted. For example, based on an analysis of temperature data from the procedure instrument data as compared to a procedure temperature criterion for desired patient body temperature, the example control system can be configured to transmit a command to cause the actuator 908 to change or maintain a desired operation setting. The example control system can be used to establish a control loop that causes actuator 908 to modify or maintains a setting of the drive medical instrument (in this example, a heater/chiller) based on a temperature sensor measurement, which in turn helps to maintain or modify a temperature of a patient's body.

In non-limiting examples, the actuator 908 could be an element or component that is configured to change the setting of a drive medical instrument, based on a mechanical, electrical, or other control mechanism. As an example, the actuator 908 could be configured to exert control by using application of a voltage to change the set point of the drive medical instrument (in an example, a heater/chiller), e.g., for controlling the temperature. The control system could form a control loop using an actuator 908 mounted to cause a modification or other adjustment to a user accessible control on a drive medical instrument, and also could be configured to allow the control system to function even if a drive medical instrument has no facility for receiving or acting on control instructions sent by wired or wireless means, or cannot be retrofitted for receiving or acting on such control instructions. The actuator 908 could be an element configured to change a setting on the user control panel of the heater/chiller 910, e.g., actuator 908 could include a servo-motor with an element that turns the knob on a manual control for the heater/chiller 910, to maintain temperature within upper and lower bounds of a procedure criterion. As another example, the actuator 908 could exert control by using voltage to change the settings of the drive medical instrument (in this example, a heater/chiller), e.g., for controlling the temperature. In another example, the actuator 908 could be configured as a servo motor that presses a button on the user control panel of the instrument to increase or decrease the set point temperature of the heater/chiller 910. The example control systems according to the principles herein could be configured to implement a user control panel actuator approach that allows medical instruments (including drive medical instruments and sense medical instruments) to be used in control systems irrespective of the design specifications or capabilities of the original instrument as manufactured, and allows the medical operators themselves to perform the setup and operation of the control loop using the control system, including by automating the procedure.

FIG. 9B shows an example control system that includes a sense medical instrument (sensor Si 902), image recording systems 904 and 904′ to capture images of an instrument display 906 or a data recording object 906′, for display of sensor data, and a drive medical instrument 910. An example implementation of the control system includes performing sensor measurements using the sensor 51 902, using the image recording system 904 to capture images of the instrument display 906 (for the sensor 51 902) and/or the image recording system 904′ to capture images of the data recording object 906′, and using the data transmission component of each respective image recording systems 904 and 904′ to transmit the image data to the console 120. The processing device 122 of the console 120 is used to perform the image analysis (such as data extraction) to provide procedure instrument data and/or the procedure event data, and using the procedure instrument data and/or the procedure event data from the data analysis to control the drive medical instrument 910 directly and/or to cause the actuator 908 to control the drive medical instrument. As a non-limiting example, the actuator 908 can be used to control the drive medical instrument 910 (such as a heater/chiller), to controlling the operating setting if the example control system determines based on the analysis that the sensor measurement and/or data recorded on the data recording object, is to be adjusted. For example, based on an analysis of temperature data from the procedure instrument data as compared to a procedure temperature criterion for desired patient body temperature, the example control system can be configured to transmit a command to cause the actuator 908 to change or maintain a desired operation setting. The example control system can be used to establish a control loop that causes actuator 908 to modify or maintains a setting of the drive medical instrument. In an example where the drive medical instrument is a heater/chiller, the control system can be used to monitor a temperature sensor measurement, which in turn can be used in an analysis to cause the drive medical instrument to maintain or modify a setting to control the temperature of a patient's body.

In an example where the sense medical instrument is a blood pressure measurement instrument, the control system can be used to control blood flow in an extracorporeal circuit (drive medical instrument). In an example where the sense medical instrument is a biometric measurement instrument (such as but not limited to, for monitoring heart rate), the control system can be used to control a syringe pump (drive medical instrument) to control the amount and/or flow rate of a drug or other pharmaceutical agent, or a biologic or other biological matter, being introduced to a patient body. For example, with an amount of a vasodilator being introduced to a patient body, a measure of blow flow may change. An indication of a change in the blood flow (based on procedure instrument data and/or procedure event data) can cause the control system to modify or maintain a setting of a drive medical instrument.

The example control system of FIG. 9A and/or 9B can include a plurality of sense medical instruments, such as but not limited to the three sensors (e.g., sensor Si and optional sensors S2 and S3) coupled to various locations on the patient. As shown in FIG. 9A and 9B, the plurality of sense medical instruments (such as but not limited to S1 and optional sensors S2 and S3) can be in communication with the same instrument display, e.g., instrument display 906 or 906′, for display of sensor data from all three sensors S1, S2, and S3. In this example, a user can indicate each region of interest of the image of the instrument display that includes the measurement data from each respective sense medical instrument. In another example, some or all of the plurality of sense medical instruments can be in communication with different instrument displays. In an example, the median or average value from the plurality of sense medical instruments can be used to determine the input for the drive medical instrument (in this example, a heater/chiller) to cause a change in operation or maintain a state of operation (e.g., for modifying or maintaining the temperature setting of operation).

The example control system of FIGS. 9A and/or 9B can be configured to control (i.e., to change or maintain) a setting of operation of the drive medical instrument at a response time that is medically desirable and practical. The example control system are configured to collect images that indicate the changes on the displays of the sense medical instrument, and can be configured to collect the images at a timescale compatible with the medical instrument displays. For example, the medical instrument displays are configured to refresh at a rate compatible with the ability of medical operators to observe and respond to them (including medical operator attention rate). In another example, the control system of FIGS. 9A and/or 9B can be controlled with response times on the order of several seconds, or tens of seconds, or one or more minutes, or longer. The example control system can be configured to response on a timescale that it might take a medical operator to read a temperature shift in the body and respond by adjusting a chiller setting. The example control system does not need to exhibit a response on the order of fractions of a second (such as might be found in noise cancellation systems), although such a rapid-response system may be used. The response time of the control system also may be configured based on a response time limitation of the drive medical instrument relative to the body response time. For example, a response time may be set depending on how rapidly a drive medical instrument is designed to be adjusted or activated by a medical operator, or how rapidly the body can be changed by the actions of the drive medical instrument. For example, the temperature of the body can be slow to adjust (e.g., on the order of minutes to hours) when driven by a water blanket, because thermal conduction is slow relative to body mass using practical temperature differences. In this example, the response time of the control loop implemented using the control system can be set based on the timescale of the effect of the drive medical instrument. In another example, an electrical shock may be applied to the patient (e.g., using a defibrillator) as fast as the amplifier can be triggered, and the response time of the control loop implemented using the control system can be set based on the response time for acquiring the procedure instrument data or procedure event data that triggers the electrical signal to apply the shock.

In various non-limiting examples, the actuator can be configured to apply an electrical, mechanical, force, or other actuating effect to the drive medical instrument, to cause the drive medical instrument to maintain or change an operation setting, based on the image data collected using the image recording system. In an example, the actuator can be a mechanical component coupled to the drive medical instrument to turn a dial to control the drive medical instrument. For example, the actuator may be calibrated such that a specified change in the amount of the setting of the actuator (based on instructions from the console) can correspond to a specific change in the setting of the drive medical instrument (such as but not limited to a 30-degree rotation of a dial of the drive medical instrument). In another example, an image capture device of the image recording system may be mounted relative to the display of the drive medical instrument to collect images of the protocol instrument data corresponding to the target setting of the drive medical instrument. In this example, the actuator can be adjusted to achieve the desired setting of the drive medical instrument based on the collected images. In an example, the image recording system can include an image capture component coupled to the drive medical instrument, to capture image data representative of a setting of the drive medical instrument. This image data can be analyzed as described herein to cause a change or maintain signal to be transmitted to the actuator and/or the drive medical instrument, as yet another example of a control loop.

A user of the example systems, such as but not limited to a physician, could set richer control settings based on the back-and-forth between the sensor measurements and the actuator via the control system.

In some examples according to the principles herein, the image recording system can include one or more transmission unit(s) in communication with the image capture device(s), to transmit data representative of captured image(s) to at least one processing unit, such as but not limited to at least one processing unit of the console. The at least one processing unit of the console can be configured to receive the data representative of the captured images, associate a timestamp with them, and performs other procedures described hereinabove, including storing the data representative of the captured images and associated timestamp to a database, and/or retrieving the same from the data representative of the captured images and associated timestamp from the database. The at least one processing unit of the console also can be configured to send instructions to the image recording system to cause the image capture device(s) to capture the image(s) according to the logging instructions, such as but not limited to, at regular time intervals. The example systems, methods, and apparatus according to the principles herein can be implemented as a distributed network of processing unit(s). In such examples, the processing unit(s) can be configured to implement identical or different procedural tasks. The functionality of the example systems, methods, and apparatus according to the principles herein can be distributed in different ways.

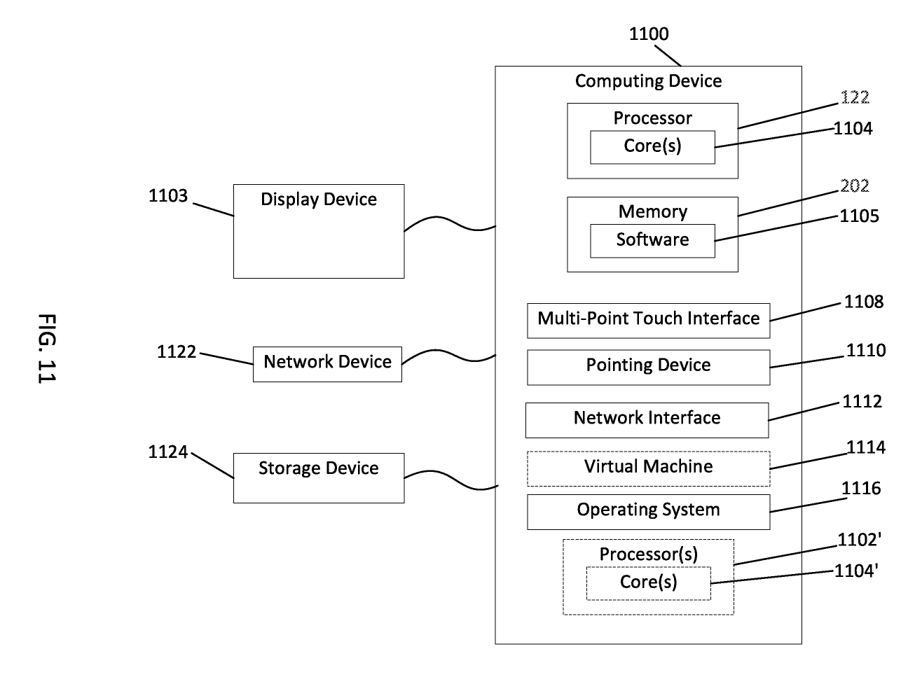

FIG. 10 illustrates a network diagram depicting a system 1000 suitable for a distributed implementation of the examples described herein. The system 1000 can include a network 1001, a console 1003, an image recording system 1007, an analytics engine 1011, and a database 1015. The image recording system includes image capture device(s) 1009, as described hereinabove. As will be appreciated, the analytics engine 1011 can be local or remote servers, and various distributed or centralized configurations may be implemented, and in some examples a single server can be used. In examples, console 1003 and the analytics engine 1011 can include one or more modules, which can implement one or more of the processes described herein, or portions thereof, with reference to FIGS. 2B-9. For example, the console 1003 can include a graphical representation module 1005 configured to generate a graphical representation of the results of one or more computations, such as but not limited to the at least one medical procedure parameter, or the at least one alert parameter. The graphical representation module 1005 also can be used to generate a graphical representation of metrics related to the medical data. The graphical representations can illustrate information relating to the at least one medical procedure parameter and/or the at least one alert parameter. The analytics engine 1011 can include a data computation module 1013 configured to compare the at least one medical procedure parameter and/or the at least one alert parameter using the medical data, and to create data comparisons or analytics to be incorporated into the graphical representations generated by the graphical representation module 1005. The console 1003, image recording system 1007, and analytics engine 1011 can communicate with each other and with the database 1015 to transmit and receive messages, perform database queries, and implement the processes described above.

In examples, the console 1003 may include a display unit 1010, which can be used to display a GUI 1002 to a user of the console 1003, such that the user can view displayed images, the indicators to select the region(s) of interest, and the graphical representations, as described above. The console 1003 may include, but is not limited to, smart phones, tablets, ultrabooks, netbooks, laptops, computers, general purpose computers, Internet appliances, hand-held devices, wireless devices, portable devices, wearable computers, cellular or mobile phones, portable digital assistants (PDAs), desktops, multi-processor systems, microprocessor-based or programmable consumer electronics, game consoles, set-top boxes, network PCs, mini-computers, smartphones, tablets, netbooks, and the like. The console 1003 may include some or all components described hereinbelow in relation to computing device 1100 shown in FIG. 11. The console 1003 may connect to network 1001 via a wired or wireless connection. The console 1003 may include one or more applications such as, but not limited to, a web browser and the like.

In examples, the user can interact with the console 1003 using a keyboard, mouse, gamepad controller, voice commands, or non-touch gestures recognizable by the user electronic device. In alternative examples, the console 1003 can be a mobile device, such as a smartphone, or tablet.

In examples, the console 1003, image recording system 1007, analytics engine 1011, and database 1015 may be in communication with each other via a communication network 1001. The communication network 1001 may include, but is not limited to, the Internet, an intranet, a LAN (Local Area Network), a WAN (Wide Area Network), a MAN (Metropolitan Area Network), a wireless network, an optical network, and the like. In one example, the console 1003, image recording system 1007, and analytics engine 1011 can transmit instructions to each other over the communication network 1001. In examples, the medical data (including region of interest markers, procedure instrument data, procedure event data, and other data) can be stored at database 1015 and received at the analytics engine 1011.

FIG. 11 is a block diagram of an exemplary computing device 1100 that can be used in the performance of any of the example methods according to the principles described herein. The computing device 1100 includes one or more non-transitory computer-readable media for storing one or more computer-executable instructions (such as but not limited to software or firmware) for implementing any example method according to the principles described herein. The non-transitory computer-readable media can include, but are not limited to, one or more types of hardware memory, non-transitory tangible media (for example, one or more magnetic storage disks, one or more optical disks, one or more USB flashdrives), and the like.

For example, memory 202 included in the computing device 1100 can store computer-readable and computer-executable instructions or software (1105) for implementing examples and programmed to perform processes described above in reference to FIGS. 2A-9. The computing device 1100 also includes processing device 122 and associated core 1104, and optionally, one or more additional processor(s) 1102′ and associated core(s) 1104′ (for example, in the case of computer systems having multiple processors/cores), for executing computer-readable and computer-executable instructions or software stored in the memory 202 and other programs for controlling system hardware. Processing device 122 and processor(s) 1102′ can each be a single core processor or multiple core (1104 and 1104′) processor.

Virtualization can be employed in the computing device 1100 so that infrastructure and resources in the computing device can be shared dynamically. A virtual machine 1114 can be provided to handle a process running on multiple processors so that the process appears to be using only one computing resource rather than multiple computing resources. Multiple virtual machines can also be used with one processor.

Memory 202 can be non-transitory computer-readable media including a computer system memory or random access memory, such as DRAM, SRAM, EDO RAM, and the like. Memory 202 can include other types of memory as well, or combinations thereof.

A user can interact with the computing device 1100 through a display device 1103 (including console 1003), such as a touch screen display or computer monitor, which can display one or more graphical user interfaces (GUIs) 1002 that can be provided in accordance with examples. The computing device 1100 can include or be in communication with an image recording system 1007 for capturing the images of instrument displays or objects as described hereinabove. The computing device 1100 can also include other I/O devices for receiving input from a user, for example, a keyboard or any other suitable multi-point touch interface 1108, a pointing device 1110 (e.g., a pen, stylus, mouse, or trackpad). The user input can include an indication of the boundary of the region of interest. The keyboard or other suitable multi-point touch interface 1108 and the pointing device 1110 can be coupled to the display device 1103. The computing device 1100 can include other suitable conventional I/O peripherals.

The computing device 1100 can also include or be in communication with one or more storage devices 1124, such as a hard-drive, CD-ROM, or other non-transitory computer readable media, for storing data and computer-readable instructions and/or software, such as a graphical representation module 1005 and a data computation module 1013 that can implement examples of the methods and systems as taught herein, or portions thereof. Exemplary storage device 1124 can also store one or more databases 1015 for storing any suitable information required to implement examples. The databases can be updated by a user or automatically at any suitable time to add, delete, or update one or more items in the databases. Exemplary storage device 1124 can store one or more databases 1015 for storing the medical data (including region of interest markers), procedure instrument data, procedure event data, and any other data/information used to implement examples of the systems and methods described herein.

The computing device 1100 can include a network interface 1112 configured to interface via one or more network devices 1122 with one or more networks, for example, Local Area Network (LAN), Wide Area Network (WAN) or the Internet through a variety of connections including, but not limited to, standard telephone lines, LAN or WAN links (for example, 802.11, T1, T3, 56 kb, X.25), broadband connections (for example, ISDN, Frame Relay, ATM), wireless connections, controller area network (CAN), or some combination of any or all of the above. The network interface 1112 can include a built-in network adapter, network interface card, PCMCIA network card, card bus network adapter, wireless network adapter, USB network adapter, modem or any other device suitable for interfacing the computing device 1100 to any type of network capable of communication and performing the operations described herein. Moreover, the computing device 1100 can be any computer system, such as a workstation, desktop computer, server, laptop, handheld computer, tablet computer (e.g., the iPad® tablet computer), mobile computing or communication device (e.g., the iPhone® communication device), or other form of computing or telecommunications device that is capable of communication and that has sufficient processor power and memory capacity to perform the operations described herein.

The computing device 1100 can run any operating system 1116, such as any of the versions of the Microsoft® Windows® operating systems, the different releases of the Unix and Linux operating systems, any version of the MacOS® for Macintosh computers, any embedded operating system, any real-time operating system, any open source operating system, any proprietary operating system, any operating systems for mobile computing devices, or any other operating system capable of running on the computing device and performing the operations described herein. In examples, the operating system 1116 can be run in native mode or emulated mode. In an example, the operating system 1116 can be run on one or more cloud machine instances.

In describing example embodiments, specific terminology is used for the sake of clarity. For purposes of description, each specific term is intended to at least include all technical and functional equivalents that operate in a similar manner to accomplish a similar purpose. Additionally, in some instances where a particular example embodiment includes a plurality of system elements, device components or method steps, those elements, components or steps can be replaced with a single element, component or step. Likewise, a single element, component or step can be replaced with a plurality of elements, components or steps that serve the same purpose. Moreover, while example embodiments have been shown and described with references to particular embodiments thereof, those of ordinary skill in the art will understand that various substitutions and alterations in form and detail can be made therein without departing from the scope of the disclosure. Further still, other aspects, functions and advantages are also within the scope of the disclosure.

Example flowcharts are provided herein for illustrative purposes and are non-limiting examples of methods. One of ordinary skill in the art will recognize that example methods can include more or fewer steps than those illustrated in the example flowcharts, and that the steps in the example flowcharts can be performed in a different order than the order shown in the illustrative flowcharts.

What is claimed is:

1. An operating location logging system for use in a complex medical procedure, the system comprising:
an image recording system configured to capture an image of at least a portion of at least one of a display of an instrument or a data recording object, the image recording system comprising:
an image capture device;
a mounting device coupled to the image capture device, the mounting device being configured to mount the image recording system relative to the data recording object or the display of the instrument; and
a data transmission component;
a console;
a database; and
at least one processing device programmed to:
cause the data transmission component to transmit image data representative of at least one image of a plurality of images captured using the image capture device;
associate a timestamp with the at least one image of the plurality of images, each timestamp being representative of a time of capture of the at least one image;
associate operator level data with at least one region of interest of the at least one image, the operator level data comprising at least one of: a procedure event data, a procedure criterion, a procedure step, a region of interest identifier, or an instrument identifier; and
store the image data associated with the respective at least one region of interest of the at least one image and the associated respective timestamp in the database as patient medical data;
wherein the console is configured to:
display the at least one image of the plurality of images on a display unit; and
render a graphical user interface comprising at least one of:
A) a first interactive user interface element configured for receiving a user indication of previously stored at least one region of interest and associated operator level data to be retrieved; or
B) a second interactive user interface element configured for receiving a user indication of previously stored medical data to be retrieved; or
C) a third interactive user interface element configured for receiving a user indication of the at least one region of interest of the at least one image of the plurality of images; and
a fourth interactive user interface element for receiving the operator level data corresponding to the at least one region of interest of the at least one image.

2. The system of claim 1, wherein the console comprises a housing, and wherein the at least one processing device is housed in the housing.

3. The system of claim 1, wherein the at least one processing device is further programmed to:
compute at least one alert parameter using the procedure criterion; and
generate at least one graphical representation of the at least one alert parameter.

4. The system of claim 3, wherein the at least one processing device is further programmed to render the graphical representation of the at least one alert parameter.

5. The system of claim 1, wherein the at least one processing device is further programmed to extract the procedure instrument data from the image data representative of the at least one region of interest of the at least one image of the portion of the display of the instrument.

6. The system of claim 5, wherein the instrument is at least one of: a blood pump, a temperature monitor attached to a temperature probe, an ECG system, a drug infusion system, or a fluoroscopy system.

7. The system of claim 1, wherein the at least one processing device is further programmed to extract the procedure event data from the image data representative of the at least one region of interest of the at least one image of the data recording object.

8. The system of claim 7, wherein the data recording object is at least one of: a clipboard or a logbook.

9. The system of claim 1, wherein the image capture device and the data transmission component are housed in a single housing.

10. The system of claim 1, wherein the at least one image is captured using the image capture device based at least in part on logging instructions.

11. The system of claim 1, wherein the console is configured as a mobile phone, a portable digital assistant, a laptop computer, a tablet computer, or a wireless portable device.

12. The system of claim 1, wherein the image recording system further comprises a linkage coupling the mounting device to the image capture device, the linkage being sufficiently resilient to minimize drift of the image capture device from a targeted orientation relative to at least one of the data recording object or the display of the instrument over a specified period of time T, and wherein a principal dimension of the image capture device is such that the image capture device minimally obstructs a view of the at least one object or display of the instrument at the target orientation.

13. A method for logging a complex medical procedure using an operating location logging system, the method comprising:
capturing a plurality of images of at least a portion of at least one of a display of an instrument or a data recording object using an image capture device of an image recording system, the image recording system comprising:
an image capture device;
a mounting device coupled to the image capture device, the mounting device being configured to mount the image recording system relative to the data recording object or the display of the instrument; and
a data transmission component;
using a console comprising a display unit to display at least one image of the plurality of images; and
using at least one processing device to:
cause the data transmission component to transmit image data representative of the at least one image of the plurality of images;
associate a timestamp with the at least one image of the plurality of images, each timestamp being representative of a time of capture of the at least one image;
display the at least one image to a display of a console;
render a graphical user interface to the display of the console, the graphical user interface comprising at least one of:
A) a first interactive user interface element configured for receiving a user indication of previously stored at least one region of interest and associated operator level data to be retrieved; or
B) a second interactive user interface element configured for receiving a user indication of previously stored patient medical data to be retrieved; or
C) a third interactive user interface element configured for receiving a user indication of the at least one region of interest of the at least one image of the plurality of images; and
a fourth interactive user interface element for receiving the operator level data corresponding to the at least one region of interest of the at least one image;
associate operator level data with at least one region of interest of the at least one image, the operator level data comprising at least one of: a procedure event data, a procedure criterion, a procedure step, a region of interest identifier, or an instrument identifier; and
store the image data associated with the respective at least one region of interest of the at least one image and the associated respective timestamp in a database as the patient medical data.

14. The method of claim 13, wherein the console comprises a housing, and wherein the at least one processing device is housed in the housing.

15. The method of claim 13, further comprising using the at least one processing device to:
compute at least one alert parameter using the procedure criterion; and
generate at least one graphical representation of the at least one alert parameter.

16. The method of claim 15, wherein the at least one processing device is further programmed to render the graphical representation of the at least one alert parameter.

17. The method of claim 13, wherein the at least one processing device is further programmed to extract the procedure instrument data from the image data representative of the at least one region of interest of the at least one image of the portion of the display of the instrument.

18. The method of claim 17, wherein the instrument is at least one of: a blood pump, a temperature monitor attached to a temperature probe, an ECG system, a drug infusion system, or a fluoroscopy system.

19. The method of claim 13, wherein the at least one processing device is further programmed to extract the procedure event data from the image data representative of the at least one region of interest of the at least one image of the data recording object.

20. The method of claim 19, wherein the data recording object is at least one of: a clipboard or a logbook.

21. The method of claim 13, wherein the image capture device and the data transmission component are housed in a single housing.

22. The method of claim 13, wherein the at least one image is captured using the image capture device based at least in part on logging instructions.

23. The method of claim 13, wherein the console is a mobile phone, a portable a digital assistant, a laptop computer, a tablet computer, or a wireless portable device.

24. The method of claim 13, wherein the image recording system further comprises a linkage coupling the mounting device to the image capture device, the linkage being sufficiently resilient to minimize drift of the image capture device from a targeted orientation relative to at least one of the data recording object or the display of the instrument over a specified period of time T, and wherein a principal dimension of the image capture device is such that the image capture device minimally obstructs a view of the at least one object or display of the instrument at the target orientation.

25. An operating location logging system for use in a complex medical procedure, the system comprising:
an image recording system configured to capture an image of at least a portion of at least one of a display of an instrument or a data recording object, the image recording system comprising:
an image capture device;
a mounting device coupled to the image capture device, the mounting device being configured to mount the image recording system relative to the data recording object or the display of the instrument; and
a data transmission component;
a console;
a database; and
at least one processing device programmed to:
cause the data transmission component to transmit image data representative of at least one image of a plurality of images captured using the image capture device;
associate a timestamp with the at least one image of the plurality of images, each timestamp being representative of a time of capture of the at least one image;
associate operator level data with at least one region of interest of the at least one image, the operator level data comprising at least one of: a procedure event data, a procedure criterion, a procedure step, a region of interest identifier, or an instrument identifier;
extract procedure instrument data and/or procedure event data from the image data corresponding to at least one region of interest, the procedure instrument data and/or the procedure event data comprising one or more of: alphanumeric data, data indicative of a graphical feature associated with an image object in the region of interest, or data indicative of a notation made in the region of interest; and
store the extracted procedure instrument data and/or the extracted procedure event data and the associated respective timestamp in the database as patient medical data;
wherein the console is configured to:
display the at least one image of the plurality of images on a display unit; and
render a graphical user interface comprising at least one of:
A) a first interactive user interface element configured for receiving a user indication of previously stored at least one region of interest and associated operator level data to be retrieved; or
B) a second interactive user interface element configured for receiving a user indication of previously stored patient medical data to be retrieved; or
C) a third interactive user interface element configured for receiving a user indication of the at least one region of interest of the at least one image of the plurality of images; and
a fourth interactive user interface element for receiving the operator level data corresponding to the at least one region of interest of the at least one image.

26. The system of claim 25, wherein the console comprises a housing, and wherein the at least one processing device is housed in the housing.

27. The system of claim 25, wherein the at least one processing device is further programmed to:
compute at least one alert parameter using the procedure criterion; and
generate at least one graphical representation of the at least one alert parameter.

28. The system of claim 25, wherein the at least one processing device is further programmed to render the graphical representation of the at least one alert parameter.

29. The system of claim 25, wherein the at least one processing device is further programmed to extract the procedure instrument data from the image data representative of the at least one region of interest of the at least one image of the portion of the display of the instrument.

30. The system of claim 29, wherein the instrument is at least one of: a blood pump, a temperature monitor attached to a temperature probe, an ECG system, a drug infusion system, or a fluoroscopy system.

31. The system of claim 25, wherein the at least one processing device is further programmed to extract the procedure event data from the image data representative of the at least one region of interest of the at least one image of the data recording object.

32. The system of claim 31, wherein the data recording object is at least one of: a clipboard or a logbook.

33. The system of claim 25, wherein the image capture device and the data transmission component are housed in a single housing.

34. The system of claim 25, wherein the image capture device comprises a mounted camera.

35. The system of claim 25, wherein the at least one image is captured using the image capture device based at least in part on logging instructions.

36. The system of claim 25, wherein the console is a mobile phone, a portable digital assistant, a laptop computer, a tablet computer, or a wireless portable device.

37. The system of claim 25, wherein the at least one processing device is programmed to perform an optical character recognition to extract the procedure instrument data and/or the procedure event data.

38. The system of claim 25, wherein the image recording system further comprises a linkage coupling the mounting device to the image capture device, the linkage being sufficiently resilient to minimize drift of the image capture device from a targeted orientation relative to at least one of the data recording object or the display of the instrument over a specified period of time T, and wherein a principal dimension of the image capture device is such that the image capture device minimally obstructs a view of the at least one object or display of the instrument at the target orientation.

39. The system of claim 25, comprising analyzing the extracted procedure instrument data and/or the extracted procedure event data to determine an occurrence of a medically-significant event in connection with the complex medical procedure.

40. A method for logging a complex medical procedure using an operating location logging system, the method comprising:
capturing a plurality of images of at least a portion of at least one of a display of an instrument or a data recording object using an image capture device of an image recording system, the image recording system comprising:
an image capture device;
a mounting device coupled to the image capture device, the mounting device being configured to mount the image recording system relative to the data recording object or the display of the instrument; and
a data transmission component;
using a console comprising a display unit to display at least one image of the plurality of images; and
using at least one processing device to:
cause the data transmission component to transmit image data representative of the at least one image of the plurality of images;
associate a timestamp with the at least one image of the plurality of images, each timestamp being representative of a respective time of capture of the at least one image;
render a graphical user interface to the display of the console, the graphical user interface comprising at least one of:
A) a first interactive user interface element configured for receiving a user indication of previously stored at least one region of interest and associated operator level data to be retrieved; or
B) a second interactive user interface element configured for receiving a user indication of previously stored patient medical data to be retrieved; or
C) a third interactive user interface element configured for receiving a user indication of the at least one region of interest of the at least one image of the plurality of images; and
a fourth interactive user interface element for receiving the operator level data corresponding to the at least one region of interest of the at least one image;
associate the operator level data with the respective at least one region of interest of the at least one image of the plurality of images;
extract procedure instrument data and/or procedure event data from the image data corresponding to the at least one region of interest, the procedure instrument data and/or the procedure event data comprising one or more of: alphanumeric data, data indicative of a graphical feature associated with an image object in the region of interest, or data indicative of a notation made in the region of interest; and
store the extracted procedure instrument data and/or the extracted procedure event data and the associated respective timestamp in a database as the patient medical data.

41. The method of claim 40, wherein the console comprises a housing, and wherein the at least one processing device is housed in the housing.

42. The method of claim 40, further comprising using the at least one processing device is further programmed to:
compute at least one alert parameter using the procedure criterion; and
generate at least one graphical representation of the at least one alert parameter.

43. The method of claim 42, wherein the at least one processing device is further programmed to render the graphical representation of the at least one alert parameter.

44. The method of claim 40, wherein the at least one processing device is further programmed to extract the procedure instrument data from the image data representative of the at least one region of interest of the at least one image of the portion of the display of the instrument.

45. The method of claim 44, wherein the instrument is at least one of: a blood pump, a temperature monitor attached to a temperature probe, an ECG system, a drug infusion system, or a fluoroscopy system.

46. The method of claim 40, wherein the at least one processing device is further programmed to extract the procedure event data from the image data representative of the at least one region of interest of the at least one image of the data recording object.

47. The method of claim 46, wherein the data recording object is at least one of: a clipboard or a logbook.

48. The method of claim 40, wherein the image capture device and the data transmission component are housed in a single housing.

49. The system of claim 40, wherein the image capture device comprises a mounted camera.

50. The method of claim 40, wherein the at least one image is captured using the image capture device based at least in part on logging instructions.

51. The method of claim 40, wherein the console is a mobile phone, a portable a digital assistant, a laptop computer, a tablet computer, or a wireless portable device.

52. The method of claim 40, wherein the at least one processing device is programmed to perform an optical character recognition to extract the procedure instrument data and/or the procedure event data.

53. The method of claim 40, wherein the image recording system further comprises a linkage coupling the mounting device to the image capture device, the linkage being sufficiently resilient to minimize drift of the image capture device from a targeted orientation relative to at least one of the data recording object or the display of the instrument over a specified period of time T, and wherein a principal dimension of the image capture device is such that the image capture device minimally obstructs a view of the at least one object or display of the instrument at the target orientation.

54. The method of claim 40, further comprising analyzing the extracted procedure instrument data and/or the extracted procedure event data to determine an occurrence of a medically-significant event in connection with the complex medical procedure.

55. An operating location logging system for use in a complex medical procedure, comprising:
an image recording system configured to capture an image of a portion of at least one of a display of an instrument or a data recording object, the image recording system comprising:
an image capture device;
a mounting device coupled to the image capture device, the mounting device being configured to mount the image recording system relative to the data recording object or the display of the instrument; and
a data transmission component;
a console;
a database; and
at least one processing device programmed to:
cause the data transmission component to transmit image data representative of at least one image of a plurality of images captured using the image capture device;
associate a timestamp with the at least one image of the plurality of images, each timestamp being representative of a time of capture of the at least one image;
associate operator level data with at least one region of interest of the at least one image, the operator level data comprising at least one of: a procedure event data, a procedure criterion, a procedure step, a region of interest identifier, or an instrument identifier;
extract procedure instrument data and/or procedure event data from the image data corresponding to the at least one region of interest, the procedure instrument data and/or the procedure event data comprising one or more of: alphanumeric data, data indicative of a graphical feature associated with an image object in the region of interest, or data indicative of a notation made in the region of interest;
compute at least one medical procedure parameter using the procedure instrument data and/or the operator level data, and the associated timestamp;
generate at least one graphical representation of the at least one medical procedure parameter; and
store the at least of the extracted procedure instrument data, or the extracted procedure event data, or the at least one medical procedure parameter, and the associated respective timestamp in the database as patient medical data;
wherein the console is configured to:
display the at least one image of the plurality of images on a display unit; and
render a graphical user interface comprising at least one of:
A) a first interactive user interface element configured for receiving a user indication of previously stored at least one region of interest and associated operator level data to be retrieved; or
B) a second interactive user interface element configured for receiving a user indication of previously stored patient medical data to be retrieved; or
C) a third interactive user interface element configured for receiving a user indication of the at least one region of interest of the at least one image of the plurality of images; and
a fourth interactive user interface element for receiving the operator level data corresponding to the at least one region of interest of the at least one image; and
display a rendering of the at least one graphical representation of the at least one medical procedure parameter.

56. The system of claim 55, wherein the console comprises a housing, and wherein the at least one processing device is housed in the housing.

57. The system of claim 55, wherein the at least one processing device is further programmed to:
compute at least one alert parameter using the procedure criterion; and
generate at least one graphical representation of the at least one alert parameter.

58. The system of claim 57, wherein the at least one processing device is further programmed to render the graphical representation of the at least one alert parameter.

59. The system of claim 55, wherein the at least one processing device is further programmed to extract the procedure instrument data from the image data representative of the at least one region of interest of the at least one image of the portion of the display of the instrument.

60. The system of claim 59, wherein the instrument is at least one of: a blood pump, a temperature monitor attached to a temperature probe, an ECG system, a drug infusion system, or a fluoroscopy system.

61. The system of claim 55, wherein the at least one processing device is further programmed to extract the procedure event data from the image data representative of the at least one region of interest of the at least one image of the data recording object.

62. The system of claim 61, wherein the data recording object is at least one of: a clipboard or a logbook.

63. The system of claim 55, wherein the image capture device and the data transmission component are housed in a single housing.

64. The system of claim 55, wherein the at least one image is captured using the image capture device based at least in part on logging instructions.

65. The system of claim 55, wherein the console comprises a mobile phone, portable digital assistant, laptop computer, tablet computer, or a wireless portable device.

66. The system of claim 55, wherein the at least one processing device is programmed to perform an optical character recognition to extract the procedure instrument data and/or the procedure event data.

67. The system of claim 55, wherein the console is configured to receive user input to specify a type of graphical representation, and wherein the at least one processing device is programmed to compute the at least one graphical representation of the at least one medical parameter based at least in part on the specified graphical representation.

68. The system of claim 67, wherein the graphical representation comprises at least one of: a bar graph, a pie chart, a line plot, point, a color pattern, a heat map, a round ideogram, or a coloration gradient.

69. The system of claim 67, wherein the graphical representation is a timeline comprising a plurality of interactive procedure event elements, each interactive procedure event element being associated with a timestamp.

70. The system of claim 55, wherein the at least one processing device is programmed to compute projected values of the at least one medical parameter as a function of time based at least in part on the extracted alphanumeric data.

71. The system of claim 70, wherein the at least one processing device is programmed to compute the compute at least one alert parameter corresponding to the at least one medical parameter based at least in part on the projected values.

72. The system of claim 55, wherein the image recording system further comprises a linkage coupling the mounting device to the image capture device, the linkage being sufficiently resilient to minimize drift of the image capture device from a targeted orientation relative to at least one of the data recording object or the display of the instrument over a specified period of time T, and wherein a principal dimension of the image capture device is such that the image capture device minimally obstructs a view of the at least one object or display of the instrument at the target orientation.

73. The system of claim 55, further comprising analyzing the extracted procedure instrument data and/or the extracted procedure event data to determine an occurrence of a medically-significant event in connection with the complex medical procedure.

74. The system of claim 55, wherein the at least one image of the plurality of images comprises a first image associated with at least a first timestamp and a second image associated with at least a second timestamp, and wherein the at least one processing device is programmed to:
extract first procedure instrument data and/or first operator level data from the image data corresponding to a first region of interest of the first image;
extract second procedure instrument data and/or second operator level data from the image data corresponding to a second region of interest of the second image;
compute at least one composite medical procedure parameter using at least one of:
the first extracted procedure instrument data, the first extracted operator level data, the second extracted procedure instrument data, or the second extracted operator level data, and the associated first timestamp or the associated second timestamp; and
generate at least one graphical representation of the at least one composite medical procedure parameter.

75. The system of claim 74, wherein the first image and the second image are a same image of the plurality of images.

76. The system of claim 74, wherein the first image and the second image are different images of the plurality of images.

77. The system of claim 74, wherein the console is configured to receive user input to specify a type of graphical representation, and wherein the at least one processing device is configured to compute the at least one graphical representation of the at least one composite medical parameter based at least in part on the specified graphical representation.

78. The system of claim 77, wherein the graphical representation comprises at least one of: a bar graph, a pie chart, a line plot, point, a color pattern, a heat map, a round ideogram, or a coloration gradient.

79. The system of claim 77, wherein the graphical representation is a timeline comprising a plurality of interactive procedure event elements, each interactive procedure event element being associated with a timestamp.

80. The system of claim 74, wherein the at least one processing device is configured to compute projected values of the at least one medical parameter as a function of time based at least in part on the extracted alphanumeric data.

81. The system of claim 80, wherein the at least one processing device is configured to compute the compute at least one alert parameter corresponding to the at least one composite medical parameter based at least in part on the projected values.

82. A method for logging a complex medical procedure using an operating location logging system, the method comprising:
capturing a plurality of images of at least a portion of at least one of a display of an instrument or a data recording object using an image capture device of an image recording system, the image recording system comprising:
the image capture device;
a mounting device coupled to the image capture device, the mounting device being configured to mount the image recording system relative to the data recording object or the display of the instrument; and
a data transmission component;
using a console comprising a display unit to display at least one image of the plurality of images; and
using at least one processing device to:
cause the data transmission component to transmit image data representative of the at least one image of the plurality of images;
associate a timestamp with the at least one image of the plurality of images, each timestamp being representative of a respective time of capture of the at least one image;
render a graphical user interface to the display of the console, the graphical user interface comprising at least one of:
A) a first interactive user interface element configured for receiving a user indication of previously stored at least one region of interest and associated operator level data to be retrieved; or
B) a second interactive user interface element configured for receiving a user indication of previously stored patient medical data to be retrieved; or
C) a third interactive user interface element configured for receiving a user indication of the at least one region of interest of the at least one image of the plurality of images; and
a fourth interactive user interface element for receiving the operator level data corresponding to the at least one region of interest of the at least one image;
associate the operator level data with the respective at least one region of interest of the at least one image of the plurality of images;
extract procedure instrument data and/or procedure event data from the image data corresponding to the at least one region of interest, the procedure instrument data and/or the procedure event data comprising one or more of: alphanumeric data, data indicative of a graphical feature associated with an image object in the region of interest, or data indicative of a notation made in the region of interest;
compute at least one medical procedure parameter using the procedure instrument data and/or the operator level data, and the associated timestamp;
generate at least one graphical representation of the at least one medical procedure parameter;
store the at least of the extracted procedure instrument data, or the extracted procedure event data, or the at least one medical procedure parameter, and the associated respective timestamp in a database as patient medical data; and
cause the display unit of the console to display a rendering of the at least one graphical representation of the at least one medical procedure parameter.

83. The method of claim 82, wherein the console comprises a housing, and wherein the at least one processing device is housed in the housing.

84. The method of claim 82, further comprising using the at least one processing device to:
compute at least one alert parameter using the procedure criterion; and
generate at least one graphical representation of the at least one alert parameter.

85. The method of claim 82, wherein at least one processing device is further programmed to render the graphical representation of the at least one alert parameter.

86. The method of claim 82, wherein the at least one processing device is further programmed to extract the procedure instrument data from the image data representative of the at least one region of interest of the at least one image of the portion of the display of the instrument.

87. The method of claim 86, wherein the instrument is at least one of: a blood pump, a temperature monitor attached to a temperature probe, an ECG system, a drug infusion system, or a fluoroscopy system.

88. The method of claim 82, wherein the at least one processing device is further programmed to extract the procedure event data from the image data representative of the at least one region of interest of the at least one image of the data recording object.

89. The method of claim 88, wherein the data recording object is at least one of: a clipboard or a logbook

90. The method of claim 82, wherein the image capture device and the data transmission component are housed in a single housing.

91. The method of claim 82, wherein the at least one image is captured using the image capture device based at least in part on logging instructions.

92. The method of claim 82, wherein the console comprises a mobile phone, portable digital assistant, laptop computer, tablet computer, or a wireless portable device.

93. The method of claim 82, wherein the at least one processing device is programmed to perform an optical character recognition to extract the procedure instrument data and/or the procedure event data.

94. The method of claim 82, wherein the console is configured to receive user input to specify a type of graphical representation, and wherein the at least one processing device is programmed to compute the at least one graphical representation of the at least one medical parameter based at least in part on the specified graphical representation.

95. The method of claim 94, wherein the graphical representation comprises at least one of: a bar graph, a pie chart, a line plot, point, a color pattern, a heat map, a round ideogram, or a coloration gradient.

96. The method of claim 94, wherein the graphical representation is a timeline comprising a plurality of interactive procedure event elements, each interactive procedure event element being associated with a timestamp.

97. The method of claim 82, wherein the at least one processing device is programmed to compute projected values of the at least one medical parameter as a function of time based at least in part on the extracted alphanumeric data.

98. The method of claim 97, wherein the at least one processing device is programmed to compute the compute at least one alert parameter corresponding to the at least one medical parameter based at least in part on the projected values.

99. The system of claim 82, wherein the image recording system further comprises a linkage coupling the mounting device to the image capture device, the linkage being sufficiently resilient to minimize drift of the image capture device from a targeted orientation relative to at least one of the data recording object or the display of the instrument over a specified period of time T, and wherein a principal dimension of the image capture device is such that the image capture device minimally obstructs a view of the at least one object or display of the instrument at the target orientation.

100. The system of claim 82, further comprising analyzing the extracted procedure instrument data and/or the extracted procedure event data to determine an occurrence of a medically-significant event in connection with the complex medical procedure;

101. The method of claim 82, wherein the at least one image of the plurality of images comprises a first image associated with at least a first timestamp and a second image associated with at least a second timestamp, and wherein the at least one processing device is programmed to:
extract first procedure instrument data and/or first operator level data from the image data corresponding to a first region of interest of the first image;
extract second procedure instrument data and/or second operator level data from the image data corresponding to a second region of interest of the second image;
compute at least one composite medical procedure parameter using at least one of:
the first extracted procedure instrument data, the first extracted operator level data, the second extracted procedure instrument data, or the second extracted operator level data, and the associated first timestamp or the associated second timestamp; and
generate at least one graphical representation of the at least one composite medical procedure parameter.

102. The method of claim 101, wherein the first image and the second image are a same image of the plurality of images.

103. The method of claim 101, wherein the first image and the second image are different images of the plurality of images.

104. The method of claim 101, wherein the console is configured to receive user input to specify a type of graphical representation, and wherein the at least one processing device is configured to compute the at least one graphical representation of the at least one composite medical parameter based at least in part on the specified graphical representation.

105. The method of claim 104, wherein the graphical representation comprises at least one of: a bar graph, a pie chart, a line plot, point, a color pattern, a heat map, a round ideogram, or a coloration gradient.

106. The method of claim 104, wherein the graphical representation is a timeline comprising a plurality of interactive procedure event elements, each interactive procedure event element being associated with a timestamp.

107. The method of claim 101, wherein the at least one processing device is configured to compute projected values of the at least one medical parameter as a function of time based at least in part on the extracted alphanumeric data.

108. The method of claim 107, wherein the at least one processing device is configured to compute the compute at least one alert parameter corresponding to the at least one composite medical parameter based at least in part on the projected values.

109. The method of claim 108, wherein the at least one processing device is programmed to compute the compute at least one alert parameter corresponding to the at least one medical parameter based at least in part on the projected values.

110. A control system for use in a complex medical procedure, the system comprising:
an image recording system configured to capture an image of at least a portion of a display of a sense medical instrument, the sense medical instrument being in communication with at least a first portion of a body, and the image recording system comprising:
an image capture device;
a mounting device coupled to the image capture device, the mounting device being configured to mount the image recording system relative to the display of the sense medical instrument; and
a data transmission component;
a drive medical instrument in communication with at least a second portion of the body; and
at least one processing device programmed to:
cause the data transmission component to transmit image data representative of at least one image of a plurality of images captured using the image capture device;
extract procedure instrument data from the image data corresponding to a region of interest of at least one image of the plurality of images, the procedure instrument data comprising data representative of at least one measurement of the sense medical instrument;
compute a control signal based at least in part on the extracted procedure instrument data to cause the drive medical instrument to maintain or change an operation setting; and
transmit the control signal to the drive medical instrument.

111. The system of claim 110, further comprising a console, wherein the console is configured to:
display at least one image of the plurality of images on a display unit; and
render a graphical user interface comprising an interactive user interface element configured for receiving a user indication of the at least one region of interest of the at least one image of the plurality of image.

112. The system of claim 110, wherein transmitting the control signal to the drive medical instrument comprises using a wired component and/or a wireless component, configured to transmit an analog signal and/or a digital signal to control the drive medical instrument.

113. The system of claim 110, further comprising an actuator in communication with the drive medical instrument, wherein transmitting the control signal to the drive medical instrument comprises causing the actuator to actuate a component of the drive medical instrument.

114. The system of claim 113, wherein the actuator comprises a mechanical component coupled to the drive medical instrument to control the drive medical instrument.

115. The system of claim 113, wherein the image recording system comprises a second image capture component coupled to the drive medical instrument, and wherein the second image capture device is configured to collect image data representative of a setting of the drive medical instrument.

116. The system of claim 115, wherein the at least one processing device is further programmed to use the image data representative of the setting of the drive medical instrument to verify the setting applied by the actuator to the drive medical instrument.

117. The system of claim 110, wherein the drive medical instrument is in communication with an application device, and wherein the application device is in communication with the at least the second portion of the body.

118. The system of claim 110, wherein the image recording system further comprises a linkage coupling the mounting device to the image capture device, the linkage being sufficiently resilient to minimize drift of the image capture device from a targeted orientation relative to the display of the sense medical instrument over a specified period of time T, and wherein a principal dimension of the image capture device is such that the image capture device minimally obstructs a view of the display of the sense medical instrument at the target orientation.

119. The system of claim 110, wherein the image recording system is configured to capture an image of a portion of a data recording object, and wherein the at least one processing device is programmed to extract procedure event data from the image data corresponding to a region of interest of at least one image of the plurality of images.

120. A method of controlling a drive medical instrument using a control system, the method comprising:
capturing a plurality of images of at least a portion of a display of a sense medical instrument, the sense medical instrument being in communication with at least a first portion of a body, and the image recording system comprising:
an image capture device;
a mounting device coupled to the image capture device, the mounting device being configured to mount the image recording system relative to the display of the sense medical instrument; and
a data transmission component;
coupling a drive medical instrument in communication with at least a second portion of the body; and
using at least one processing device to:
cause the data transmission component to transmit image data representative of at least one image of a plurality of images captured using the image capture device;
extract procedure instrument data from the image data corresponding to a region of interest of the at least one image, the procedure instrument data comprising data representative of at least one measurement of the sense medical instrument;
compute a control signal based at least in part on the procedure instrument data to cause the drive medical instrument to maintain or change an operation setting; and
transmit the control signal to the drive medical instrument.

121. The method of claim 120, wherein a console is configured to:
display at least one image of the plurality of images on a display unit; and
render a graphical user interface comprising an interactive user interface element configured for receiving a user indication of the at least one region of interest of the at least one image of the plurality of image.

122. The method of claim 120, wherein transmitting the control signal to the drive medical instrument comprises using a wired component and/or a wireless component, configured to transmit an analog signal and/or a digital signal to control the drive medical instrument.

123. The method of claim 120, further comprising an actuator in communication with the drive medical instrument, wherein transmitting the control signal to the drive medical instrument comprises causing the actuator to actuate a component of the drive medical instrument.

124. The method of claim 123, wherein the actuator comprises a mechanical component coupled to the drive medical instrument to control the drive medical instrument.

125. The method of claim 123, wherein the image recording system comprises a second image capture component coupled to the drive medical instrument, and wherein the second image capture device is configured to collect image data representative of a setting of the drive medical instrument.

126. The method of claim 125, wherein the at least one processing device is further programmed to use the image data representative of the setting of the drive medical instrument to verify the setting applied by the actuator to the drive medical instrument.

127. The method of claim 120, wherein the drive medical instrument is in communication with an application device, and wherein the application device is in communication with the at least the second portion of the body.

128. The method of claim 120, wherein the image recording system further comprises a linkage coupling the mounting device to the image capture device, the linkage being sufficiently resilient to minimize drift of the image capture device from a targeted orientation relative to the display of the sense medical instrument over a specified period of time T, and wherein a principal dimension of the image capture device is such that the image capture device minimally obstructs a view of the display of the sense medical instrument at the target orientation.

129. The method of claim 120, wherein the image recording system is configured to capture an image of a portion of a data recording object, and wherein the at least one processing device is programmed to extract procedure event data from the image data corresponding to a region of interest of at least one image of the plurality of images.
